SEQUENCE LISTING (8 sequences)

SEQ ID NO: 1 - protein, 361 aa, Homo sapiens
GEGLDFPEYDGVDRVINVNAKNYKNVFKKYEVLALLYHEPPEDDKASQRQFEMEELILELAAQVLEDKGVGFGLVDSEKDAAVAKKLGLTEVDSMYVFKGDEVIEYDGEFSADTIVEFLLDVLEDPVELIEGERELQAFENIEDEIKLIGYFKSKDSENYKAFEDAAEEFHPYIPFFATFDSKVAKKLTLKLNEIDFYEAFMEEPVTIPDKPNSEEEIVNFVEEHRRSTLRKLKPESMYETWEDDMDGIHIVAFAEEADPDGFEFLETLKAVAQDNTENPDLSIIWIDPDDFPLLVPYWEKTFDIDLSAPQIGVVNVTDADSVWMEMDDEEDLPSAEELEDWLEDVLEGEINTEDDDDDDD

SEQ ID NO: 2 - protein, 350 aa, Homo sapiens
GLNFPTYDGKDRVVSLSEKNFKQVLKKYDLLCLYYHEPVSSDKVTQKQFQLKEIVLELVAQVLEHKAIGFVMVDAKKEAKLAKKLGFDEEGSLYILKGDRTIEFDGEFAADVLVEFLLDLIEDPVEIISSKLEVQAFERIEDYIKLIGFFKSEDSEYYKAFEEAAEHFQPYIKFFATFDKGVAKKLSLKMNEVDFYEPFMDEPIAIPNKPYTEEELVEFVKEHQRPTLRRLRPEEMFETWEDDLNGIHIVAFAEKSDPDGYEFLEILKQVARDNTDNPDLSILWIDPDDFPLLVAYWEKTFKIDLFRPQIGVVNVTDADSVWMEIPDDDDLPTAEELEDWIEDVLSGKIN

SEQ ID NO: 3 - protein, 353 aa, Danio rerio
GLDFPEYDGKDRVHQLTAKNYKSVMKKYDVMVIYLHKPVGEDRMARKQFEVEELALELAAQVLDGLDDEDIGFGLVDSKKDRAVAKKLGMLEVDSIYIFADDEIIEYDGALAADTLLEFLYDVIEDPVEIISNDRELKGFHNIEEDMKLMGFFKSNKSPYFIEYDDAAEEFHPFIKFFATFEPKIAKKLNLKMNEVDFYEPFMDKPVTIPGKPYMEDDIINFIEDHDRPTLRKLEPHSMYEIWEDDINGQHIVAFAEESDPDGYEFLEILKEVAQENTENPELSIIWIDPDDFPLMVPYWEKTFGIDLSSPQIGVVDVENADSVWMEMDDEEHMPTADQLDAWIEDVMTGNIN

SEQ ID NO: 4 - protein, 354 aa, Haliaeetus leucocephalus
GDGLDFPTYDGLDRVLPVTLKNYKAMLKRFPVLALLHHRPSQGDRAAQRHSEMEELILELAAQVLEDKGVGFGLVDSEKDAAVAKKLGLTEEDSIYVFKEDEVIEYDGELAADTLVEFLLDVLEDPVEFIEGDHELQAFENIEDDPKLIGYFKNKDSEHFKAFEQAAEEFHPYIPFFATFDSKAAKKLTLKLNEIDFYKPFMEEPLTIPDQPNSKEEIMAFMEEHKRATLRKLKPESMYETWEDDMDGIHIVAFAEEDDPDGFEFLEILKDVARDNTDNPDLSILWIDPEDFPLLIPYWEKTFNIDLSRPQIGVVNVTDADSVWLEMADEDDLPSPAELEEWIEDVLAGEINTE

SEQ ID NO: 5 - protein, 352 aa, Gekko japonicus
GLDFPEYDGIDRVVDINAKNYKAVLKKFEVLALLYHEPVEDTKASQRQFEMEELILELAAQVLEDKGVGFGLVDSEKDAAVAKKLGLTEEDSVYVFKEDEVIEYDGEFSADTLVEFLLDVLEDPVEFIEGDHELEAFENIEDEPKLIGFFKNEDSEHYKAYLDAAEEFHPYIPFFVTFDSKVAKKLSLKLNEIDYYEPFMEEPVTIPDKPNSEEEIMQFLEEHKRPTLRKLQPDSMYETWEDDIDGIHIVAFAEEDDPDGYEFLEILKDVAQDNTDNPDLSIIWIDPEDFPLLIPYWEKTFDIDLNRPQIGVVNVTDADSIWLEMDDEDDLPSADELEDWLEDVLEGEINTE

SEQ ID NO: 6 - protein, 351 aa, Salvelinus alpinus
KGLEFPRYDGNDRVIDINDKNYKKAMKKYSILCLLYHKPIPDGKELQKQHQMTEMVLELAAQVMEEKEIGFGMVDSHEDVKVAKKLGLVEEGSVYVFKGDRVIEFDGLLSADTLVEFLLDLLEEPVEVIGNTLELRAFDRMEEDIRLIGYFKNDESEHYHAFKEAAEQFQPYIRFFATFEKSVAKELTLKMNEVDFYEPFMEEPVTVPDRPNSEEEIVAFVTEHRRPTLRKLRAEDMFETWEDDLEGIHVVAFAEEEDPDGYEFLELLKEVARDNTHHPGLSIIWIDPDDFPLLIPYWEKTFHVDLFKPQIGVVNVTDADSIWLEIDEQDLPTAQELEDWIEDVLSGKVNT

SEQ ID NO: 7 - protein, 360 aa, Caenorhabditis elegans
LGYPDLEYDGFDRTEVLTEKNFNRTVFAEDTKSVVFFNDVEEDDSELDQYECFLQLSAQIMTKRGYNFYTVNTTKEHRLRKQEEVEKGEDTIHVYKDGYKIEYNGVRDPETFVSWLMDIPDDPVTIINDEHDLEEFENMDDECVRIIGYFEPGSVALKEFEEAAEDFMGEIEFFAVVTSKWARKVGLKRVGEVQMRRPFEEDPLFAPTSADTEEEFEDWVEKNKEPVMQKLTLDNYFNLWRDPEEEERMILAFVDEETREGRAMKRLLDKIADENSEHAGTLEIILVDPDEFPLMVDVWEDMFGIDIEEGPQIGLIDISEKEGIWFDMSQVNLDDPKKHSDSNFEALQSWIDQILSGSIS

SEQ ID NO: 8 - protein, 379 aa, Homo sapiens
TTEITSLDTENIDEILNNADVALVNFYADWCRFSQMLHPIFEEASDVIKEEFPNENQVVFARVDCDQHSDIAQRYRISKYPTLKLFRNGMMMKREYRGQRSVKALADYIRQQKSDPIQEIRDLAEITTLDRSKRNIIGYFEQKDSDNYRVFERVANILHDDCAFLSAFGDVSKPERYSGDNIIYKPPGHSAPDMVYLGAMTNFDVTYNWIQDKCVPLVREITFENGEELTEEGLPFLILFHMKEDTESLEIFQNEVARQLISEKGTINFLHADCDKFRHPLLHIQKTPADCPVIAIDSFRHMYVFGDFKDVLIPGKLKQFVFDLHSGKLHREFHHGPDPTDTAPGEQAQDVASSPPESSFQKLAPSEYRYTLLRDRDEL

(19) United States
(12) Patent Application Publication
(10) Pub. No.: US 2021/0107956 A1
(43) Pub. Date: Apr. 15, 2021

(54) ULTRASENSITIVE DETECTION PLATFORM USING LUMINESCENT METALS AND USES THEREOF

(71) Applicants: James Alan BROZIK, Pullman, WA (US); Chul-Hee KANG, Pullman, WA (US)

(72) Inventors: James Alan BROZIK, Pullman, WA (US); Chul-Hee KANG, Pullman, WA (US)

(73) Assignee: WASHINGTON STATE UNIVERSITY, Pullman, WA (US)

(21) Appl. No.: 16/500,482

(22) PCT Filed: Apr. 3, 2018
(86) PCT No.: PCT/US2018/025852

§ 371 Date: Oct. 3, 2019

Related U.S. Application Data
(60) Provisional application No. 62/480,970, filed on Apr. 3, 2017.

(51) Int. Cl. C07K 14/47; G01N 33/58; A61K 49/00

(52) U.S. Cl. CPC C07K 14/4728; A61K 49/0019; A61K 49/0056; G01N 33/582

(57) ABSTRACT

Polypeptides which comprises a plurality of luminescent metal binding sites and constructs comprising the polypeptides and bound luminescent metals are provided. When the constructs are exposed to suitable wavelengths of energy, the bound luminescent metals emit characteristic wavelengths of light. Thus, the constructs are used as luminescent tracking molecules e.g. as probes, markers, reporters, etc., and to deliver cargo to targeted cells or tissues.

### BACKGROUND OF THE INVENTION

### Field of the Invention

The present disclosure provides metal binding proteins/polypeptides which chelate luminescent metals, and constructs comprising the metal binding proteins/polypeptides and one or more chelated luminescent metals. In particular, the luminescent metals are selected so that, in the constructs, they produce desired wavelengths of light in response to irradiation at suitable frequencies.

### SEQUENCE LISTING

This application includes as the Sequence Listing the complete contents of the accompanying text file “Sequence.txt”, created Mar. 27, 2018, containing 65,536 bytes, hereby incorporated by reference.

### Discussion of the Background Art

Reporter molecules that utilize luminescence are frequently used for a variety of purposes. For example, they are used to monitor gene expression and in high-sensitivity biochemical assays in both research and medicine where they increasingly replace radioisotopes. This change has been driven partly by the increasing expense of radioisotope disposal and partly by the need to find more rapid and convenient assay methods.

The desire to perform biochemical assays in situ in living cells and whole animals has driven researchers toward protein-based luminescence and fluorescence. For example, the extensive use of firefly luciferase for ATP assays, aequorin and obelin as calcium reporters, Vargula luciferase as a neurophysiological indicator, and the Aequorea green fluorescent protein as a protein tracer and pH indicator show the potential of luminescence-based methods in research laboratories. Such technology also directly impacts medicine and biotechnology. For example, Aequorea GFP is employed to mark cells in murine model systems and as a reporter in high throughput drug screening, and Renilla luciferase is under development for use in diagnostic platforms.

However, the currently available reporter genes have certain drawbacks that limit their use. For example, a frequently encountered limitation is the requirement for introduction of a substrate. Other drawbacks include, for example, the large size of certain proteins, which means that expression of reporter-fusion proteins can be difficult.

Another useful strategy is to label a protein with a fluorescent tag to enable subsequent detection and localization in intact cells. Fluorescence labeling has generally been achieved by purifying proteins and covalently conjugating them to reactive derivatives of organic fluorophores. However, in these methods, the stoichiometry and locations of dye attachment are often difficult to control and careful repurification of the proteins is usually necessary. A further problem is introducing the labeled proteins into a cell, which often involves microinjection techniques or methods of reversible permeabilization to introduce the proteins through the plasma membrane.

A molecular biological alternative to fluorescent-tagged proteins was made possible by the cloning of Aequorea victoria GFP. Light-stimulated GFP fluorescence is species-independent and does not require any cofactors, substrates, or additional gene products from A. victoria, permitting GFP detection in living cells other than A. victoria. While extremely useful, GFP continues to have severe limitations both in terms of performance and spectroscopic range. Specifically, GFP photobleachs after a few seconds of exposure and the protein is prone to misfolding upon cellular expression, rendering the protein non-fluorescent. This limits the sensitivity and linear range of GFP as a probe molecule.

It is evident that new developments in reporter molecule technology are needed.

### SUMMARY OF THE INVENTION

Other features and advantages of the present invention will be set forth in the description of invention that follows, and in part will be apparent from the description or may be learned by practice of the invention. The invention will be realized and attained by the compositions and methods particularly pointed out in the written description and claims hereof.

Disclosed herein are novel constructs which comprise i) a protein/polypeptide which comprises at least one, and generally a plurality of, binding sites for luminescent metals; and ii) one or more types of chelated luminescent metals. When the constructs are exposed to suitable wavelengths of electromagnetic energy (e.g., ultraviolet light, visible light, infrared light, etc.), the bound metals absorb energy and emit luminescence at one or more characteristic wavelengths. The wavelengths which are emitted depend on which metals or combinations of metals are selected for inclusion in a particular construct. Thus, the absorption and emission characteristics of the constructs are “tunable” and can be pre-selected to correspond to a desired outcome. The constructs do not photobleach or photoblink and find use as luminescent tracking or reporter molecules in a variety of applications e.g. as probes, markers, reporters, etc. as well as for therapeutic applications.

The foregoing and other objects, features, and advantages of the present disclosure will become more apparent from the following detailed description, which proceeds with reference to the accompanying figures.

It is an object of this invention to provide synthetic constructs comprising, i) a genetically engineered, recombinant (e.g. synthetic) polypeptide comprising a plurality of luminescent metal binding sites, and ii) a plurality of chelated luminescent metals bound to the binding sites. In some aspects, the chelated luminescent metals are rare earth metals or actinides. In other aspects, the chelated luminescent metals are selected from the group consisting of Ce, Dy, Er, Eu, Gd, Ho, La, Lu, Nd, Pr, Pm, Sm, Sc, Tb, Tm, Yb, and Y. In some aspects, the actinides are U or Th. In other aspects, the construct comprises at least two different types of chelated luminescent metals. In further aspects, the plurality of luminescent metal binding sites comprise i) one or more high affinity luminescent metal-binding sites; and ii) one or more medium affinity luminescent metal-binding sites. In additional aspects, the genetically engineered, recombinant polypeptide is a modified calcium binding polypeptide, and in yet other aspects, the genetically engineered, recombinant polypeptide is a fusion polypeptide. In some aspects, the fusion polypeptide comprises a targeting moiety and/or a half-life expanding moiety. In additional aspects, the targeting moiety is an antibody or antigen binding portion thereof.

The invention also provides a genetically engineered, recombinant (e.g. synthetic) polypeptide comprising a plurality of luminescent metal binding sites. In some aspects, the plurality of luminescent metal binding sites comprise i) one or more high affinity luminescent metal-binding sites; and ii) one or more medium affinity luminescent metal-binding sites. In other aspects, the genetically engineered, recombinant polypeptide is a modified calcium binding polypeptide. In further aspects, the genetically engineered, recombinant polypeptide of claim 11, wherein the genetically engineered, recombinant polypeptide is a fusion polypeptide. In additional aspects, the fusion polypeptide comprises a targeting moiety and/or a half-life expanding moiety, and in yet further aspects, the targeting moiety is an antibody or antigen binding portion thereof. The invention also provides nucleic acids encoding the genetically engineered, recombinant polypeptides, as well as plasmids comprising one or more of the nucleic acids, and cells comprising one or more of the plasmids.

The invention also comprises a detection method, comprising: combining a sample with one or more polypeptides or proteins each having chelated thereto one or more luminescent metals; binding a molecule of interest in said sample with said one or more polypeptides or proteins; exciting said one or more luminescent metals with electromagnetic energy; and detecting luminescence from said one or more luminescent metals after said step of exciting. In some aspects, one or more of the method steps occurs on a chip or in a microwell device. In other aspects, one or more of the steps are performed as part of an ELISA assay. In some aspects, the sample is selected from the group consisting of serum, plasma, blood, saliva, cerebrospinal fluid, urine, sputum, joint fluid, body cavity fluid, whole cells, cell extracts, tissue, biopsy material, aspirates, exudates, slide preparations, fixed cells, solid tumor cells, blood tumor cells, environmental samples, forensic samples, homeland security-related samples and chemical samples. In additional aspects, the molecule of interest is a protein, an amino acid, a peptide, a nucleic acid, carbohydrate, lipid, vitamin, hemoglobin, explosive chemicals or remnants thereof, poisons, virus, bacteria or any target molecules in medical diagnostic assay, an anti-terrorism assay target, or a forensic assay target. In yet further aspects, the step of detecting is performed by Forster Resonance Energy Transfer (FRET), enzyme linked immunosorbent assay (ELISA) testing, flow cytometry, fluorescent correlation spectrometry or single particle microscopy.

The invention also provides a method of detecting an analyte located in the body of a subject, comprising administering to the subject a composition comprising a construct comprising, i) a genetically engineered, recombinant (e.g. synthetic) polypeptide comprising a plurality of luminescent metal binding sites, and ii) a plurality of chelated luminescent metals bound to the binding sites; irradiating the subject with electromagnetic energy to excite the one or more luminescent metals in the construct; and detecting luminescence from the one or more luminescent metals after the step of irradiating. In some aspects, the subject is a cancer patient and the analyte is a tumor cell marker.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A and B. Excitation spectra (light gray curves) and phosphorescence spectra (dark grey curves). FIG. 1A are spectra of Variant-615m and FIG. 1B are spectra of Variant-544m. Spectra were carried out with a 20 μM solution at 25° C. in 10 mM HEPES buffer at pH 7.4. Both Variant-615m and Variant-544m display bright highly structured phosphorescence spectra and highly structured excitation spectra. Experiments were carried out using a photon counting luminescence spectrometer. Phosphorescence was achieved by exciting the samples at 395 nm (A) or 380 nm (B) and scanning the emission spectrometer. Excitation spectra were carried out by monitoring the luminescence at 615 nm (A) or 544 nm (B) and scanning the excitation wavelengths.

FIGS. 2A and B. Phosphorescence lifetimes carried out at 25° C. in 10 mM HEPES buffer at pH 7.4. (A) Variant −615m excited at 395 nm and monitored at 625 nm. (B) Variant −544m excited at 380 nm and monitored at 615 nm. The lifetimes were measured using a photon counting spectrometer and a chopped excitation beam. In this experiment the phosphorescence is monitored after the light was turned off by scanning a time gate on the photon counter. The time resolution of the experiment was 1 sec.

FIG. 3. Head-to-head comparison of GFP vs. Variant-615m in solution. Initially GFP has a larger luminosity but photobleaches under constant illumination. Variant-615m shows no measurable photobleaching and after ˜1 minute of illumination its luminosity surpasses that of GFP.

FIG. 4. Titration of naked variant (protein without the rare earth) with Eu(III). In this experiment the variant protein was kept at 112 nM in 10 mM HEPES at 25° C. The curve was fit to a titration model that included 4 strong binding sites and 36 weaker sites. The K_{d}s are given in the figure above.

FIG. 5. Phosphorescence spectra Variant-615m (black curve) and Variant-615d (gray curve). Variant −615d is engineered to have twice the number of rare-earth binding sites as Variant-615m, and Variant-615m is roughly ½ the size of Variant-615d. Spectra were carried out with a 20 M solution at 25° C. in 10 mM HEPES buffer at pH 7.4.

FIGS. 6A and B. Storage of Variant-615m at 4° C. (FIG. 6A) and at 25° C. (FIG. 6B). In these experiments Variant-615m was prepared as a 40 μM solution in 10 mM HEPES and allowed to sit for 6 days on the bench top or in the refrigerator. Over the six days aliquots were taken from the solutions and analyzed using phosphorescence spectroscopy. The sample stored in the refrigerator displays little to no protein degradation (FIG. 6A) while samples warmed to room temperature and left on the bench top displayed significant degradation by day 4 (FIG. 6B).

FIG. 7. Phosphorescence micrograph of E. coli. that are expressing Variant-615m. This experiment was carried out with an Olympus IX7l fluorescence microscope using a band-pass filter that cover the 395 nm excitation band of Variant −615m and a 475 long-pass emission filter. The exposure time was 350 ms.

FIG. 8. Luminescence spectrum of lyophilized Variant-980m containing Y=3, Yb⁺³ and Eu₊₃. Material was excited with 2 photons of near-IR light at 980 nm.

### DETAILED DESCRIPTION

This disclosure generally relates to polypeptides/proteins which chelate luminescent metals to form polypeptide-metal constructs. When the metals are bound to the polypeptides and exposed to wavelengths of energy that match their electron excitation energies, they absorb the energy and emit luminescence that is detectable at one or more wavelengths or wavelength ranges. The absorption and emission wavelengths depend on and are characteristic of the particular metal ions that are bound. Constructs comprising the polypeptides plus bound metals are therefore useful as tracking molecules e.g. as probes, markers, reporters, etc., as well as for a variety of other purposes. The disclosed constructs advantageously do not suffer from the limitations of the prior art (e.g. GFP) because, for example: the luminescence properties of the constructs are completely independent of small protein misfolds (i.e. the polypeptides fold properly, regardless of e.g. the type of cations in the surrounding milieu); the constructs do not photobleach or photoblink so the luminescence is long-lasting; and the wavelength(s) or emitted luminescence can be varied as needed, depending on which metals or combinations thereof are selected for use. Nucleic acids and vectors encoding the polypeptides are also provided as are methods of using the polypeptides and the polypeptide-metal constructs in a wide variety of applications.

The disclosure thus provides novel constructs which are used as reporter molecules that allow the visualization of cellular targets, subcellular targets and the distribution of tagged proteins and peptides, and for the quantitative/qualitative measurement of proteins and peptides, small organic/inorganic molecules of biologic/forensic/environmental significance, and for visualizing the targeted delivery of molecules of interest to a designated location such as a particular cell type, among other things. The benefits of using the constructs of the present disclosure are at least four-fold: they are safer than radioactive-based assays, they can be assayed quickly and easily, and large numbers of samples can be handled simultaneously, reducing overall handling and increasing efficiency. The novel constructs are particularly well suited for use in biological systems, for example, for detecting gene expression.

### Definitions

The following are definitions of terms that may be used in the present specification. The initial definition provided for a group or term herein applies to that group or term throughout the present specification individually or as part of another group, unless otherwise indicated. Additionally, it will be understood that any list of such candidates or alternatives is merely illustrative, not limiting, unless implicitly or explicitly understood or stated otherwise.

The term “luminescent metal” generally refers to d-block and f-block metals which emit phosphorescence or fluorescence upon exposure to suitable (characteristic) wavelengths of energy. The term encompasses transition and/or “rare earth” elements, lanthanides and actinides. A “luminescent metal ion” refers to the oxidized charged form of the metal. Those of skill in the art will recognize that it is generally the ionic form of a metal that is chelated via a coordinate covalent bond; however, herein chelation may be referred to a “metal” binding and chelated metals may be referred to as bound “metals”.

A coordinate covalent bond, also known as a dative bond or coordinate bond is a kind of 2-center, 2-electron covalent bond in which the two electrons that form the bond derive from the same atom but are shared by both the atoms. The bonding of metal ions to ligands involves this kind of interaction. In all cases the bond is a covalent bond. The prefix dipolar, dative or coordinate merely serves to indicate the origin of the electrons used in creating the bond. Herein, this bonding may be referred to as “chelation”.

The “d-block” is on the middle of the periodic table and includes elements from columns 3 through 12. These elements are also known as the transition metals because they show a transitivity in their properties i.e. they show a trend in their properties in simple incomplete d orbitals. Transition basically means d orbital lies between s and p orbitals and shows a transition from properties of s to p. The “d-block” elements are metals which exhibit two or more ways of forming chemical bonds. Because there is a relatively small difference in the energy of the different d-orbital electrons, the number of electrons participating in chemical bonding can vary. This results in the same element exhibiting two or more oxidation states, which determines the type and number of its nearest neighbors in chemical compounds. D-block elements are unified by having in their outermost electrons one or more d-orbital electrons but no p-orbital electrons. The d-orbitals can contain up to five pairs of electrons; hence, the block includes ten columns in the periodic table.

An “f-block metal” is a metal in the center-left of a 32-column periodic table or in the footnoted appendage of 18-column tables. These elements are not generally considered as part of any group. They are often called “inner transition metals” because they provide a transition between the s-block and d-block in the 6th and 7th row (period). The known f-block elements come in two series, the lanthanides of period 6 and the radioactive actinides of period 7. All are metals. Because the f-orbital electrons are less active in determining the chemistry of these elements, their chemical properties are mostly determined by outer s-orbital electrons. Consequently, there is much less chemical variability within the f-block than within the s-, p-, or d-blocks. F-block elements are unified by having one or more of their outermost electrons in the f-orbital but none in the d-orbital or p-orbital. The f-orbitals can contain up to seven pairs of electrons; hence, the block includes fourteen columns in the periodic table.

A rare-earth element (REE) or rare-earth metal (REM), as defined by IUPAC, is one of a set of seventeen chemical elements in the periodic table, specifically the fifteen lanthanides, as well as scandium and yttrium. Scandium and yttrium are considered rare-earth elements because they tend to occur in the same ore deposits as the lanthanides and exhibit similar chemical properties. Rare-earth elements are cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb) and yttrium (Y).

Electron excitation is the transfer of a bound electron to a more energetic, but still bound state. This can be done by photoexcitation (PE), where the electron absorbs a photon and gains all its energy or by electrical excitation (EE), where the electron receives energy from another, energetic electron. When an excited electron falls back to a state of lower energy, it undergoes electron relaxation. This is accompanied by the emission of a photon (radiative relaxation) or by a transfer of energy to another particle. The energy released is equal to the difference in energy levels between the electron energy states.

The term “luminescence” refers to the emission of light not caused by incandescence, i.e. luminescence is light that is not produced by heat. It can be caused by chemical reactions, electrical energy, subatomic motions, stress on a crystal, etc. and includes fluorescence, phosphorescence, chemiluminescence, and bioluminescence. The metals ions which are chelated with the constructs disclosed herein emit luminescence of one or more characteristic wavelengths when excited by a suitable wavelength of impinging light. The impinging light and the emitted light generally have different characteristic wavelengths.

The phrase “cellular luminescence” denotes luminescence when expressed in cells.

Fluorescence intermittency, or blinking, is the phenomenon of random switching between ON (bright) and OFF (dark) states of the emitter under its continuous excitation.

Photobleaching is the irreversible destruction of the fluorophore that can occur when the fluorophore is in an excited state, which leads to fading of fluorescence during observation.

“Upconversion” refers to a process in which the sequential absorption of two or more photons leads to the emission of light at a shorter wavelength than the excitation wavelength. This can occur between two or more different metal ions in the combinations of metal ions disclosed herein.

As used herein, the term “peptide” refers to a linear organic polymer comprising two or more and generally up to about 20 amino acid residues covalently bonded together in a chain. The term “polypeptide” generally refers to such a linear organic polymer, but one which comprises at least about 20 amino acid residues. Polypeptides of about 100 or more (e.g. 150, 200, 250 or more) amino acids may be referred to herein as “proteins”. However, the terms “polypeptide” and “protein” may sometimes be used interchangeably herein, with “protein” generally referring to relatively large polypeptides (e.g. >300, 400 or 500 amino acids), unless otherwise noted. Usage of these terms in the art overlaps and varies.

The term “homologues” refers to a peptide or DNA sequence where the primary molecular structure (i.e., the sequence of amino acids or nucleotides) of substantially all molecules present in the composition under consideration is identical. The term “substantially” used in the preceding sentences preferably means at least 80% by weight, more preferably at least 95% by weight, and most preferably at least 99% by weight.

An “EF hand calcium binding motif” is a helix-loop-helix structural domain or motif found in a large family of calcium-binding proteins. The EF-hand motif has a helix-loop-helix topology, much like the spread thumb and forefinger of the human hand, in which the Ca²⁺ ions are coordinated by ligands within the loop. The motif takes its name from traditional nomenclature used in describing the protein parvalbumin, which contains three such motifs and is probably involved in muscle relaxation via its calcium-binding activity. The EF-hand consists of two alpha helices linked by a short loop region (usually about 12 amino acids) that usually binds calcium ions. EF-hands also appear in each structural domain of the signaling protein calmodulin and in the muscle protein troponin-C.

The term “isolated” refers to material that is substantially or essentially free from components that normally accompany it as found in its native state (for example, a band on a gel). The isolated nucleic acids and the isolated proteins of this invention do not contain materials normally associated with their in situ environment, in particular, nuclear, cytosolic or membrane associated proteins or nucleic acids other than those nucleic acids, which are indicated.

### Metals

The metals that are used in the constructs described herein are typically f-block and/or d-block elements, or categories thereof such as rare earth metals, lanthanides, actinides, transition metals, etc., or even further subsets of these categories. The common features of metals that are suitable for use as described here include: 1) they are capable of binding to metal ion binding sites on at least one type of polypeptide; and 2) collectively they absorb energy throughout the near infrared (900 nm-1600 nm), visible (400 nm-900 nm), and ultraviolet regions (290 nm-400 nm) of electromagnetic spectrum and emit energy at wavelengths throughout the visible and near infrared at levels that are detectable, e.g. at least about 1-10 photons/sec above background and often greater than about 100,000 photons/second above background (background is typically about 1 photon/second or even less, with a “detectable” signal typically taken as 2 standard deviations above the background). In some aspects, the emitted wavelengths are in the visible range.

In some aspects, the metals that are used in the constructs are the rare earth elements Ce, Dy, Er, Eu, Gd, Ho, La, Lu, Nd, Pr, Pm, Sm, Sc, Tb, Tm, Yb, and Y. The oxidation states of the metals may be, for example, +2, +3 or +4, depending on the type of metal and the particular binding site. Thus, in some aspects, the +3 oxidation states of those elements are used, e.g. Er(III), Eu(III), Gd(III), Nd(III), Sm(III), Tb(III), Tm(III), Yb(III), and Y(III). However, the +2 oxidation states can be used as well, e.g. Er(II), Eu(II), Gd(II), Nd(II), Sm(II), Tb(II), Tm(II), Yb(II), and Y(II). In addition, 4+ oxidation states are used as follows: Ce(IV), Dy(IV), Nd(IV), Pr(IV), and Tb(IV). The elements outside the rare earths (and forms thereof) which can be used, include but are not limited to the actinides: U(IV), (UO₂)²⁺, and Th(IV). In some aspects, the metals that are used are not radioactive; in other aspects, the metals that are used are radioactive.

Examples of metals that can be used in the constructs together with their excitation and emission maxima, or ranges thereof, are as follows:

| | UV and Visible | Visible and Near IR |
|---|---|---|
| | Absorptions Bands | Luminescence Bands |
| Metal | (Approximate Wavelengths) | (Approximate Wavelengths) |
| Eu³⁺ | 276 nm, 324 nm, 337 nm, | 579 nm, 591 nm, 615 nm, |
| | 358 nm, 370 nm, 395 nm, | 690 nm, 695 nm, 700 nm |
| | 474 nm, 500 nm, 534 nm | |
| Tb³⁺ | 353 nm, 368 nm, 399 nm, | 490 nm, 544 nm, 585 nm, |
| | 421 nm, 472 nm, 483 nm, | 621 nm |
| | 490 nm | |
| Dy³⁺ | 351 nm, 365 nm, 390 nm, | 480 nm, 572 nm, 618 nm |
| | 428 nm, 450 nm, 475 nm | |
| Er³⁺ | 365 nm, 382 nm, 410 nm, | 518 nm, 527 nm, 595 nm, |
| | 481 nm, 518 nm | 550 nm, 650 nm, 675 nm, |
| | | 1520 nm |
| Nd³⁺ | 350 nm, 471 nm, 505 nm, | 1049 nm, 1180 nm |
| | 579 nm, 748 nm, 795 nm | |

Combinations of metals may also be used in a polypeptide, e.g. combinations of metals that interact in order to create luminescent upconversion probes in which infrared (IR) excitation leads to visible luminescence. It is well known in the field that many of the excited states of rare earth ions are metastable and this phenomenon allows energy to hop from one ion to the next (exciton-hopping). The variants generally contain more than one rare earth ion with a lower energy metastable state which can be efficiently populated and last for a long time at a well-defined location. This allows for a second photon to be absorbed, which creates an exciton of much higher energy—which can populate yet a third metal ion that can emit light at a frequency about twice that of the excitation beam. The close clustering of rare earth ions in the variants is uniquely suited to promote such a process. Exemplary metal combinations include but are not limited to: Eu³⁺, Y³⁺, and Yb³⁺; Er³⁺, Y³⁺, and Yb³⁺, Yb³⁺ and Tm³⁺; Y³⁺, Gd³⁺, Yb³⁺ and Tm³⁺. Alternatively, a group or plurality of polypeptides, at least some of which have bound luminescent metals which differ from the metals bound to other polypeptides in the group, may be used together as a “probe”. For example, some polypeptides in the preparation comprise bound Eu³⁺, others comprise bound Y³⁺ and yet others comprise bound Yb³⁺. When used together, upconversion occurs between the different metals in the polypeptides e.g. upon infrared excitation, leading to e.g. visible luminescence.

All metals that are used as described herein are in general readily available from commercial sources.

### Metal Binding Polypeptides

The constructs disclosed herein comprise a protein or polypeptide that comprises at least one, and usually more than one, metal ion binding site. The protein/polypeptide is generally not autofluorescent (intrinsically fluorescent). However, proteins/polypeptides that are autofluorescent are not excluded from use.

In some aspects, the polypeptides are proteins that are known to bind metals that do not fluoresce (e.g. Ca⁺²) and it has been discovered that the proteins surprisingly also bind (chelate) one or more of the fluorescent metals disclosed herein. In other aspects, the polypeptides are novel recombinant, genetically engineered variants of such proteins which differ from the parent or native form of the protein (e.g. as found in nature, “wild type”), e.g. in one or more of length, amino acid sequence, charge, solubility, Kd for metals, number of metal binding sites, etc., but which retain the ability to bind luminescent metals, i.e. the variants or modified forms have luminescent metal binding activity. The polypeptides and modified forms thereof may be monomeric or multimeric, e.g. dimeric, trimeric, etc.

For example, the polypeptides may be or may be modified forms of known calcium binding proteins, examples of which include but are not limited to: calsequestrin (e.g. cardiac or skeletal calsequestrin), parvalbumin, calmodulin (CaM), troponin-C, the prokaryotic CaM-like protein calerythrin, calmodulin, calreticulin, S100 proteins, calcineurin, annexin, vitamin D-dependent calcium-binding protein, ERp44, calbindin, TCBP-23, TCBP-27, CDPK, calcyphosin, calretinin, etc. Alternatively, the polypeptides may be modified forms of other known metal binding proteins, examples of which include but are not limited to: alcohol dehydrogenase, recoverin, reticulocalbin, squidulin, troponin C.

In some aspects, the recombinant polypeptides described herein differ from known naturally occurring metal binding proteins and/or from known genetically engineered variants thereof. For example, they may contain fewer amino acids due to the deletion of sequences from the known or natural protein, i.e. sequences which are not needed to bind rare earth metals may be removed. Such shortening (truncation) of a protein sequence may be advantageous due to ease of handling, synthesis, etc. Alternatively, portions of a protein that are removed may be substituted with heterologous sequences. A “heterologous” sequence is a sequence (amino acid or nucleic acid) which is present in a polypeptide or a nucleic acid, respectively, in which it is not found in nature e.g. sequences from another protein, or the same protein but from a different species, etc. If sequences are substituted or replaced with heterologous sequences, the resulting polypeptides may be referred to as “fusion” or “chimeric” proteins/polypeptides. In all cases, the resulting modified polypeptide retains the ability to bind at least one metal ion at a level that is sufficient to insure retention of the metal ion in the construct during use.

Alternatively, or in addition, the polypeptides may include one or more changes in primary amino acid sequence, compared to the parent protein/polypeptide, i.e. they may comprise one or more mutations. The term “mutation” carries its traditional connotation and means a change (inherited, naturally occurring or deliberately introduced via e.g. genetic engineering) in a polypeptide sequence, generally due to a mutation in an encoding nucleic acid, and is used herein in its sense as generally known to those skilled in the art. Amino acid mutations that may be introduced include but are not limited to: conservative amino acid substitutions in which an amino acid is replaced by another from a group having similar structure and/or general chemical characteristics of R (variable, side chain) groups. For example, similar amino acids may be grouped as follows: aliphatic amino acids include glycine, alanine, valine, leucine and isoleucine; hydroxyl or sulfur/selenium-containing amino acids include serine, cysteine, selenocysteine, threonine and methionine; cyclic amino acids include proline; aromatic amino acids include phenylalanine, tyrosine and tryptophan; basic amino acids include histidine, lysine and arginine; and acidic amino acids and their amides include aspartate, glutamate, asparagine and glutamine. Alternatively, non-conservative amino acid substitution are also encompasses, e.g. in which an uncharged amino acid is replaced by a charged amino acid or vice versa; or an amino acid with a bulky side chain (e.g. tryptophan, leucine) is replaced by an amino acid with a smaller side chain (alanine, glycine), etc. In addition, so-called “non-natural”, non-standard, non-coded and/or non-proteinogenic amino acids may also be used in the sequences, examples of which include but are not limited to: selenocysteine, pyrroysine, β-alanine, γ-aminobutyric acid (GABA), δ-aminolevulinic acid, 4-aminobenzoic acid (PABA), aminoisobutyric acid, dehydroalanine, norvaline, norleucine, homonorleucine, etc. The amino acids may be either D or L amino acids, and isomeric forms of amino acids are also encompassed.

In some aspects, the polypeptides may be substantially “artificial” in nature, having no comparable “parent” or natural (wild type) counterpart. For example, the polypeptides may be designed ab initio. Such polypeptides may comprise one or more known or artificially designed metal binding sites joined or spaced apart by amino acid sequences which allow the binding sites to act independently with respect to binding metal ions, i.e. the sites do not occlude each other.

### Exemplary Polypeptides

In some aspects, the polypeptides which are used in the constructs are naturally occurring metal binding proteins, or modified forms thereof, but they have previously been used only to bind non-fluorescent metals. In such aspects, the disclosure provides novel constructs which comprise the known protein, or a modified form thereof, plus at least one bound fluorescent metal ion.

In aspects of the disclosure, the known protein is calsequestrin or a modified form thereof. Exemplary calsequestrin sequences which may be used, or portions or modifications of which may be used, in the constructs described herein include but are not limited to:

(SEQ ID NO: 1)
GEGLDFPEYDGVDRVINVNAKNYKNVEKKYEVLALLYHEPPEDDKASQRQ
FEMEELILELAAQVLEDKGVGEGLVDSEKDAAVAKKLGLTEVDSMYVFKG
DEVIEYDGEFSADTIVEFLLDVLEDPVELIEGERELQAFENIEDEIKLIG
YEKSKDSENYKAFEDAAEEFHPYIPFFATFDSKVAKKLTLKLNEIDEYEA
FMEEPVTIPDKPNSEEEIVNEVEEHRRSTLRKLKPESMYETWEDDMDGIH
IVAFAEEADPDGFEFLETLKAVAQDNTENPDLSIIWIDPDDFPLLVPYWE
KTEDIDLSAPQIGVVNVTDADSVWMEMDDEEDLPSAEELEDWLEDVLEGE
INTEDDDDDDDD.

SEQ ID NO: 1 is the wild type calsequestrin sequence found in human skeletal muscle (calsequestrin-1). The homologous proteins with a significant level of sequence identity or similarity (at least about 90%, such as 91, 92, 93, 94, 95, 96, 97, 98 or 99% similarity) exist in every vertebrate's muscular tissues. There are several hundred sequences of calsequestrin which are available from various repositories. Examples of such proteins are as follows:

Human cardiac calsequestrin (calsequestrin-2)
(SEQ ID NO: 2)
GLNFPTYDGKDRVVSLSEKNFKQVLKKYDLLCLYYHEPVSSDKVTQKQFQ
LKEIVLELVAQVLEHKAIGFVMVDAKKEAKLAKKLGFDEEGSLYILKGDR
TIEFDGEFAADVLVEFLLDLIEDPVEIISSKLEVQAFERIEDYIKLIGFF
KSEDSEYYKAFEEAAEHEQPYIKFFATFDKGVAKKESLKMNEVDFYEPFM
DEPIAIPNKPYTEEELVEFVKEHQRPTLRRLRPEEMFETWEDDLNGIHIV
AFAEKSDPDGYEFLEILKQVARDNTDNPDLSILWIDPDDFPLLVAYWEKT
FKIDLFRPQIGVVNVTDADSVWMEIPDDDDLPTAELLEDWIEDVLSGKIN
Calsequestrin-1 from Danio rerio
(SEQ ID NO: 3)
GLDFPEYDGKDRVHQLTAKNYKSVMKKYDVMVIYLHKPVGEDRMARKQFE
VEELALELAAQVLDGLDDEDIGGGLVDSKKDRAVAKKLGMLEVDSIYIFA
DDEIIEYDGALAADTLLEFLYDVIEDPVEIISNDRELKGFHNIEEDMKLM
GFFKSNKSPYFIEYDDAAEEFHPFIKFFATFEPKIAKKLNLKMNEVDFYE
PFMDKPVTIPGKPYMEDDIINFIEDHDRPTLRKLEPHSMYEIWEDDINGQ
HIVAFAEESDPDGYEFLEILKEVAQENTENPELSIIWIDPDDEPLMVPYW
EKTFGIDLSSPQIGVVDVENADSVWMEMDDEEHMPTADQLDAWIEDVMTG
NIN
calsequestrin-1,from Haliaeentus leucocephalus
(SEQ ID NO: 4)
GDGEDFPTYDGLDRVLPVTLKNYKAMLKRFPVLALLHHRPSQGDRAAQRH
SEMEELILELAAQVLEDKGVGFGLVDSEKDAAVAKKLGLTEEDSIYVFKE
DEVIEYDGELAADTLVEFLLDVLEDPVEFIEGDHELQAFENIEDDPKLIG
YFKNKDSEHFKAFEQAAEEFHPYIPFFATFDSKAAKKLTLKLNEIDFYKP
FMEEPLTIPDQPNSKEEIMAFMEEHKRATLRKLKPESMYETWEDDMDGIH
IVAFAEEDDPDGFEFLEILKDVARDNTDNPDLSILWIDPEDFPLLIPYWE
KTFNIDLSRPQIGVVNVTDADSVWLEMADEDDLPSPAELEEWIEDVLAGE
INTE
calsequestrin-1 from Gekko japonicus
(SEQ ID NO: 5)
GLDFPEYDGIDRVVDINAKNYKAVLKKFEVLALLYHEPVEDTKASQRQFE
MEELILELAAQVLEDKGVGFGLVDSEKDAAVAKKLGLTEEDSVYVFKEDE
VIEYDGEFSADTLVEFLLDVLEDPVEFIEGDHELEAFENIEDEPKLIGFF
KNEDSEHYKAYLDAAEEFHPYIPFFVTFDSKVAKKLSLKLNEIDYYEPFM
EEPVTIPDKPNSEEEIMQFLEEHKRPTLRKLQPDSMYETWEDDIDGIHIV
AFAFEDDPDGYEFLEILKDVAQDNTDNPDLSIIWIDPEDFPLLIPYWEKT
FDIDLNRPQIGVVNVTDADSIWLEMDDEDDLPSADELEDWLEDVLEGEIN
TE
Calsequestrin-2 from Salvelinus alpinus
(SEQ ID NO: 6)
KGLEFPRYDGNDRVIDINDKNYKKAMKKYSILCLLYHKPIPDGKELQKQH
QMTEMVLELAAQVMEEKEIGFGMVDSHEDVKVAKKLGLVEEGSVYVFKGD
RVIEFDGLLSADTLVEFLLDLLEEPVEVIGNTLELRAFDRMEEDIRLIGY
FKNDESEHYHAFKEAAEQFQPYIRFFATFEKSVAKELTLKMNEVDFYEPF
MEEPVTVPDRPNSEEEIVAFVTEHRRPTLRKLRAEDMFETWEDDLEGIHV
VAFAEEEDPDGYEFLELLKEVARDNTHHPGLSIIWIDPDDFPLLIPYWEK
TFHVDLFKPQIGVVNVTDADSIWLEIDEQDLPTAQELEDWIEDVLSGKVN
T
Calsequestrin from Caenorhabditis elegans
(SEQ ID NO: 7)
LGYPDLEYDGFDRTEVLTEKNFNRTVFAEDTKSVVFFNDVEEDDSELDQY
ECFLQLSAQIMTKRGYNFYTVNTTKEHRLRKQEEVEKGEDTIHVYKDGYK
IEYNGVRDPETFVSWLMDIPDDPVTIINDEHDLEEFENMDDECVRIIGYF
EPGSVALKEFFEAAEDFMGEIEFFAVVTSKWARKVGLKRVGEVQMRRPFE
EDPLFAPTSADTEEEFEDWVEKNKEPVMQKLTLDNYFNLWRDPEEEERMI
LAFVDEETREGRAMKRLLDKIADENSEHAGTLEIILVDPDEFPLMVDVWE
DMFGIDIEEGPQIGLIDISEKEGIWFDMSQVNLDDPKKHSDSNFEALQSW
IDQILSGSIS

The proteins and polypeptides that are used in the present technology are not limited to calsequestrin. Examples of other types of metal binding proteins that may be used include but are in no way limited to:

Endoplasmic reticulum resident protein 44
precursor [Homo sapiens]
(SEQ ID NO: 8)
TTEITSLDTENIDEILNNADVALVNFYADWCFESQMLHPIFEEASDVIKE
EFPNENQVVFARVDCDQHSDIAQRYRISKYPTLKLFRNGMMMKREYRGQR
SVKALADYIRQQKSDPIQEIRDLAEITTLDRSKRNIIGYFEQKDSDNYRV
FERVANILHDDCAFLSAFGDVSKPERYSGDNIIYKPPGHSAPDMVYLGAM
TNEDVTYNWIQDKCVPLVREITFENGEELTEEGLPFLILFHMKEDTESLE
IFQNEVARQLISEKGTINFLHADCDKFRHPLLHIQKTPADCPVIAIDSFR
HMYVFGDFKDVLIPGKLKQFVFDLHSGKLHREFHHGPDPTDTAPGEQAQD
VASSPPESSFQKLAPSEYRYTLLRDRDEL

In some aspects, the entire family of calsequestrin proteins from a variety of organisms (human, mouse, bat, dolphin, canines, bovine, rodent, primates, reptiles, birds, amphibians, fishes, worms such as C. elegans etc.) in either cardiac or skeletal isoforms are developed into novel recombinant proteins that having specific binding affinities for specific rare earths. In other aspects, the recombinants are instead, or in addition, engineered in order accommodate greater numbers of metal ions, and thus to emit a greater number of photons than the parent molecule. For example, an engineered protein (such as an engineered calsequestrin) may emit a total number of photons that is at least about 5 to 20 times greater, than the parent molecule, e.g. about 5, 10, 15 or 20 times greater. In fact, such engineered proteins also emit from about 5-20 times (e.g. at least about 5, 10, 15, or 20 times) more photons than modified wild type GFP.

Other exemplary proteins that can be used as described herein include but are not limited to: EF-hand calcium-binding domain from calcium-binding proteins such as Aequorin, α actinin, Calbindin, Calcineurin B subunit, Calcium-binding protein from Streptomyces erythraeus, Calcium-binding protein from Schistosoma mansoni, Calcium-binding proteins TCBP-23 and TCBP-25 from Tetrahymena thermophile, Calcium-dependent protein kinases (CDPK), Calcium vector protein from amphoxius Calcyphosin, Calmodulin, Calpain, Calretinin, Calcyclin, Caltractin (centrin), Cell Division Control protein 31 (CDC31) from yeast, Diacylglycerol kinase, FAD-dependent glycerol-3-phosphate dehydrogenase, Fimbrin (plastin), Flagellar calcium-binding protein from Trypanosoma cruzi, Guanylate cyclase activating protein (GCAP), Inositol phospholipid-specific phospholipase C isozymes γ-1 and delta-1, Intestinal calcium-binding protein (ICaBPs), MIF related proteins 8 (MRP-8 or CFAG) and 14 (MRP-14), Myosin regulatory light chains, Oncomodulin, Osteonectin, Parvalbumins a and 3, Placental calcium-binding protein (18a2), Recoverins (visinin, hippocalcin, neurocalcin, S-modulin), Reticulocalbin, S-100 protein, Sarcoplasmic calcium-binding protein (SCPs), Sea urchin proteins Spec 1, Spec 2, Lps-1, Serine/threonine specific protein phosphatase rdgc from Drosophila, Sorcin V19 from hamster, Spectrin a chain, Squidulin from squid, Troponins C.

Polypeptides/proteins or functional segments thereof that can be used as described herein include those which are explicitly disclosed, and variants thereof. Such variants generally display at least about 90% or more identity and/or similarity to the disclosed sequences, when aligned according to conventional methods used in the art. Generally, the level of identity/similarity is at least about 90, 91, 92, 93, 94, 95, 96, 97, 98 or 99%, compared to a disclosed sequence. Those of skill in the art are familiar with programs for analyzing sequence identity/similarity e.g. the BLAST program at the National Institutes of Health website.

### Metal Binding Sites

The polypeptides in the disclosed constructs comprise one or more (e.g. ranging from about 1 to about 20, such as about 1, 2, 3, 4, 5, 6, 7, 8, 9, 10, 11, 12, 13, 14, 15, 16, 17, 18, 19 or 20) high affinity luminescent metal-binding sites, and generally from about 1-10 (e.g., 4) high affinity binding sites; and usually one or more (e.g. ranging from 2-100, such as about 2, 5, 10, 15, 20, 25, 30, 35, 40, 45, 50, 55, 60, 65, 70, 75, 80, 85, 90, 95, or 100) medium affinity luminescent metal-binding sites, and generally from about 2 to 60 medium affinity luminescent metal-binding sites. As used herein a “high affinity luminescent metal-binding site” exhibits a Kd in the range of from about 10 μM to about 0.1 μM (e.g. about 10, 5 or 1μ M, or about 100, 75, 50, 25, 10, 1 or 0.1 μM); and a medium affinity luminescent metal-binding site exhibits a Kd in the range of from about 10 mM to about 10 μM (e.g. about 10, 5 or 1 mM, or about 100, 75, 50, 25 or 10 μM). Generally, 4 high affinity sites are present together with at least one medium/low affinity site. The medium/low affinity sites are in equilibrium with the solutional concentration of metal ions.

In some aspects, the luminescent metal-binding sites that are utilized in the constructs are from proteins which bind metals e.g. calcium, the binding sites having been reproduced within or incorporated into a polypeptide sequence not found in the original protein. In other words, the resulting polypeptide is a chimera. Examples of metal binding sites which can be used in this manner include but are not limited to: so-called EF hand motifs, [EQ]-[DE]-G-L-[DN]-F-P-x-Y-D-G-x-D-R-V or [DE]-L-E-D-W-[LIVM]-E-D-V-L-x-G-x-[LIVM]-N-T-E-D-D-D motifs, etc.

In some aspects, the calcium-chelating sites fits the pattern PS00018 which has been generated to predict canonical EF-hand sites (e.g. see the PROSITE website). The pattern is as follows: D-{W}-[DNS]-{ILVFYW}-[DENSTG]-[DNQGHRK]-{GP}-[LIVMC]-[DENQSTAGC]-x(2)-[DE]-[LIVMFYW], wherein each amino acid position is separated from its neighbor by a hyphen, closed parentheses [ ] represent acceptable amino acids for a given position, closed parentheses { } represent the amino acids that are not allowed and a position where any amino acid is allowed is designated by x.

Constructs that Include Multiple Components

While in some aspects, the polypeptide component of the construct is a single polypeptide, in some aspects, the constructs also comprise one or more additional components, e.g. at least a second component, and possible additional components, e.g. third, fourth, fifth, etc. components, with desired functionalities. For example, the constructs may comprise a fluorescent metal binding polypeptide as described above (e.g. a “first” polypeptide) plus a second peptide or polypeptide of interest which does or does not bind fluorescent (or any other) metals e.g. the constructs may comprise a fusion protein comprising a first metal binding and a second metal-binding or non-metal binding peptide/polypeptide. The second peptide/polypeptide component may be, for example, a targeting polypeptide (such as an antibody) which is specific or selective for binding to a molecule, cell or tissue type of interest.

Examples of second components that are polypeptides include but are not limited to: an antibody (e.g. an IgG antibody and its Fc portion, an IgM antibody, etc.) or an antigen binding fragment of an antibody; cell surface binding peptides that target cancer cells (e.g. those that target cancer cell surface receptors or endothelial cell surface receptors of the neovasculature); peptides or polypeptides that bind specifically or selectively to (are ligands of) an outer membrane cell receptor; peptide sequences which facilitate entry into the cytoplasm; peptide sequences which target intracellular organelles; etc.

The term “antigen binding fragment” of an antibody, as used herein, refers to one or more fragments of an intact antibody that retain the ability to specifically binds to a given antigen or epitope. The term “epitope” means an antigenic determinant that is specifically bound by an antibody. Epitopes usually consist of surface groupings of molecules, such as amino acids and/or sugar side chains, and may be linear or have specific three-dimensional structural characteristics, as well as specific charge characteristics. Examples of antigen binding fragments or modified forms of antibodies that can be attached to a metal binding polypeptide as described herein include but are not limited to: Fab, Fab′, a F(ab)′2, a single domain antibody, a ScFv, a Sc(Fv)2, a diabody, a triabody, a tetrabody, a unibody, a minibody, a maxibody, a small modular immunopharmaceutical (SMIP), minimal recognition units consisting of the amino acid residues that mimic the hypervariable region of an antibody, an isolated complementary determining region (CDR), and fragments which comprise or consist of the VL or VH chains, and others known in the art.

In other aspects, the additional component(s) is/are not peptide based. The additional component(s) may be e.g. targeting moieties, therapeutic moieties, and the like. Examples of second components that are not polypeptides include but are not limited to: nucleic acids (e.g. DNA, RNA, DNA/RNA hybrids, aptamers, etc.); folate; transferrin; various lipids; various polymers; avidin (e.g. streptavidin); biotin; azide; alkynes; phalloidin; iodoacetamide; maleimide; various drugs (described in detail below); various half-life lengthening moieties; one or more polyethylene glycol (PEG) chains (PEGylation); albumin; GFP and its derivatives, etc.

### Nucleic Acid Sequences and Vectors

The present disclosure also encompasses nucleic acids that encode the polypeptides/proteins disclosed herein, as well as vectors comprising the nucleic acids. The term “nucleic acid” refers to a deoxyribonucleotide or ribonucleotide polymer in either single- or double-stranded form, and unless specifically limited, encompasses known analogues of natural nucleotides that hybridize to nucleic acids in a manner similar to naturally occurring nucleotides. Unless otherwise indicated, a particular nucleic acid sequence implicitly provides the complementary sequence thereof, as well as the sequence explicitly indicated. As used herein, the terms “nucleic acid” and “gene” are interchangeable, and they encompass the term “cDNA.”

The phrase “a nucleic acid sequence encoding” refers to a nucleic acid, which contains sequence information that, if translated, yields the primary amino acid sequence of a specific protein or peptide. This phrase specifically encompasses degenerate codons (i.e., different codons which encode a single amino acid) of the native sequence or sequences, which may be introduced to conform with codon preference in a specific host cell.

Exemplary encoding nucleic acid sequences are explicitly provided for the amino acid sequences disclosed herein. However, those of skill in the art will recognize that, due to the redundancy of the genetic code, more than one codon can encode the same amino acid and hence more than one nucleotide sequence can encode a particular polypeptide. Thus, this disclosure encompasses any nucleic acid which can be transcribed and/or translated to yield an amino acid sequence disclosed or described herein, as well as variants thereof.

Variants of the nucleic acids are also encompassed. Such variants generally display at least about 50% or more identical or similar to disclosed sequences, when aligned according to conventional methods used in the art (e.g. the BLAST program at the National Institutes of Health website). Generally, the level of identity/similarity is at least about 90, 91, 92, 93, 94, 95, 96, 97, 98 or 99%, compared to a disclosed sequence.

Alterations in the primary amino acid sequence of a polypeptide and/or alterations which join sequences which are not found together in nature may be introduced at the level of the encoding nucleic acid. One of skill in the art will recognize many ways of generating alterations in a given nucleic acid sequence. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques. Product information from manufacturers of biological reagents and experimental equipment also provide information useful in known biological methods. Using these techniques, it is possible to substitute at will any nucleotide in a nucleic acid that encodes any protein or polypeptide disclosed herein to produce any contemplated variant or mutant.

In some aspects, the nucleic acid sequences that encode a polypeptide are operably linked to regulatory sequences that cause the encoding sequences to be expressed, e.g. translated. The term “regulatory sequence” denotes all the non-coding elements of a nucleic acid sequence required for the correct and efficient expression of the “coding region” (i.e., the region that actually encodes the amino acid sequence of a peptide or protein), e.g., binding cites for polymerases and transcription factors, transcription and translation initiation and termination sequences, TATA box, a promoter to direct transcription, a ribosome binding site for translational initiation, polyadenylation sequences, enhancer elements. The term “operably linked” refers to functional linkage between a first nucleic acid (for example, an expression control sequence such as a promoter or an array of transcription factor binding sites) and a second nucleic acid sequence, wherein the expression control sequence directs transcription of the nucleic acid corresponding to the second sequence.

In some aspects, the nucleic acid encodes a single copy of a polypeptide. Alternatively, multiple copies of a single polypeptide may be encoded e.g. in tandem. In yet further alternatives, two or more polypeptide or peptide components that originate from different, diverse sources (e.g. different parent proteins, different species, etc.) are encoded, i.e. the amino acid sequences of the different polypeptides are heterologous and originate from different parent molecules which are not found together in nature. When multiple polypeptides are encoded, they may be encoded by a single open reading frame and thus translated as a single chimeric or fusion protein. Alternatively, two or more encoded polypeptides or polypeptide segments may be encoded with one or more intervening linking sequences so that they are joined after translation by a linking peptide sequence. Linker or linking peptide sequence are typically short (e.g. 2-20 amino acids) and typically comprise amino acids which do not interact with, or at least do not interfere with, the folding and activity of the moieties which are joined. Examples include linking peptides comprising flexible glycine and serine residues, or uncharged amino acids with relatively small side chains (e.g. alanine), and combinations of these. Such linkages may or may not be cleavable, e.g. by proteases.

The sequence of cloned genes and synthetic oligonucleotides can be verified Using, for example, the chemical degradation method. The sequence can be confirmed after the assembly of the oligonucleotide fragments into the double-stranded DNA sequence using the chain termination method for sequencing double-stranded templates. DNA sequencing may also be performed by the PCR-assisted fluorescent terminator method according to the manufacturer's instructions. Sequencing data is generally analyzed using commercially available programs.

It is expected that those of skill in the art are knowledgeable in the numerous systems available for cloning and expression of nucleic acids. As used herein, “expression” refers to transcription of nucleic acids, either without or preferably with subsequent translation. In brief summary, the expression of natural or synthetic nucleic acids is typically achieved by operably linking a nucleic acid of interest to a promoter (which is either constitutive or inducible), and incorporating the construct into an expression vector. In some aspects, the nucleic acids described herein are present in a vector. The term “vector” denotes an engineered nucleic acid construct that contains sequence elements that mediate the replication of the vector sequence and/or the expression of coding sequences present on the vector. Examples of vectors include eukaryotic and prokaryotic plasmids, viruses (for example), cosmids, phagemids, and the like. One or more selected isolated nucleic acids may be operably linked to a vector by methods known in the art.

Vectors to which selected nucleic acids are operably linked may be used to introduce these nucleic acids into host cells and mediate their replication and/or expression. The vectors are suitable for replication and integration in prokaryotes, eukaryotes, or both. Cloning vectors are useful for replicating foreign, non-native nucleic acids in host cells and expression vectors are generally used to mediate the expression of the foreign nucleic acid. Typical cloning vectors contain transcription and translation terminators, transcription and translation initiation sequences, and promoters useful for regulation of the expression of the particular nucleic acid. The vectors optionally comprise generic expression cassettes containing at least one independent terminator sequence, sequences permitting replication of the cassette in eukaryotes, or prokaryotes, or both. Some vectors are both cloning and expression vectors.

Expression of a polypeptide of interest by a vector can be enhanced by any of several known means, including inserting multiple copies of the encoding nucleic acid into a transformed host, using so-called “super promoters”, etc. In all cases, the polypeptide is expressed from a DNA sequence that is functionally inserted into a suitable vector. “Functionally inserted” means that it is inserted in proper reading frame and orientation. Typically, the encoding gene is inserted downstream from a promoter and is followed by a stop codon, although production as a hybrid protein followed by cleavage may be used, if desired.

The nucleic acids and vectors provided herein, in combination with well-known techniques for over-expressing recombinant proteins, make it possible to obtain unlimited supplies of homogeneous recombinant fluorescent metal binding proteins.

### Production and Modification of Polypeptide Components

In some aspects, the polypeptides are produced using recombinant technology, e.g. using nucleic acids that are genetically engineered to encode the polypeptides, as described in detail above. Once a nucleic acid is synthesized or isolated and inserted into a vector and cloned, one may express the nucleic acid in a variety of host cells known to those of skill in the art. For example, cells which are suitable for the expression of the nucleic acids include bacteria, yeast, filamentous fungi, insect, plant and mammalian cells, in particular cells capable of being maintained in tissue culture.

Host cells are competent or rendered competent for transformation by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextran, receptor-mediated endocytosis, electroporation and micro-injection of the DNA directly into the cells.

In other aspects, the polypeptides are produced using synthetic peptide synthesis techniques, e.g. solid-phase peptide synthesis (SPPS). In some aspects, the constructs are produced by a combination of synthetic and recombinant techniques, e.g. peptide/polypeptide components of the construct may be produced recombinantly and then chemically coupled to each other or to other components.

Alternatively, and especially if a component to be joined to a polypeptide is not peptide-based, other chemistries are used to link or conjugate the component to the peptide chain at the amino or carboxyl terminus, or at a reactive side chain (e.g. cysteine, lysine). The components may be linked (conjugated, cross-linked) together by any suitable means, such as via a “linkage group”, “linker arm”, “linker”, chemical cross linking groups, and the like. These terms refer to any of the well-known bonds or compounds used to join functional groups and which do not substantially interfere with the characteristic properties or functions of the functional groups so joined. Moieties may be attached to a variety of reactive groups on the polypeptide, e.g. primary amines, carboxyls, sulfhydryls or carbonyls. Such linkages may or may not be cleavable, e.g. by proteases, changes in pH, exposure to selected wavelength of light (photoreactive crosslinkers), etc. Commonly used crosslinking agents include but are not limited to: e.g., 1,1-bis(diazoacetyl)-2-phenylethane, glutaraldehyde, N-hydroxysuccinimide esters, for example, esters with 4-azidosalicylic acid, homo-bifunctional imidoesters, including disuccinimidyl esters such as 3,3′-dithiiobis(succinimidylpropioonate), and bifunctional maleimides such as bis-N-maleimido-1,8-octane. Derivatizing agents such as methyl-3-[(p-azidophenyl)dithio]propioimidate yield photoactivatable intermediates that are capable of forming cross links in the presence of light.

Once a luminescent metal binding polypeptide is completely synthesized, it may be isolated and/or purified as necessary for use, e.g. by known methods which include but are not limited to: centrifugation, chromatography (e.g. size exclusion, affinity, etc.), extraction, dialysis, by histidine tag purification, or by any of the many other suitable means that are known. Isolation and purification may occur as needed prior to and/or after assembly of multiple components, if applicable. Since the ultimate goal is to bind metals of choice to the polypeptide, it may be desirable to carry out one or more of the isolation and purification steps in metal free buffers to prevent unwanted binding of extraneous metals, and/or to remove such metals (e.g. with a chelating agent) prior to fluorescent metal binding. Well known spectroscopy techniques may be used to confirm the metal-binding and integrity of the protein.

Methods of Producing and/or Further Functionalizing Constructs

In some aspects what is provided is methods of producing a luminescent construct comprising a luminescent metal binding polypeptide and one or more luminescent metals. Briefly, such methods comprise: putting one or more luminescent (e.g., fluorescent) metals (e.g. in ionic form) in contact with a luminescent (e.g., fluorescent) metal binding polypeptide, and allowing the luminescent metal binding polypeptide to chelate one more metals.

In additional aspects, the polypeptides/proteins or the constructs are further functionalized. For example, they may be chemically modified with one or more reactive groups, allowing for downstream chemical attachment to other materials. For example, the constructs may be attached to a substrate for use in an assay, e.g. attached to beads, films, metals surfaces, wells of an assay plate, etc.; or attached to a miniature camera; etc. Alternatively, or in addition, as described in detail elsewhere herein, therapeutic agents may be attached to the constructs, especially to constructs which comprise targeting moieties, with the added advantage of being able to visualize the delivery of the drug to the target (cell, organ, tissue, etc.) via the long-lasting fluorescence produced by the construct.

Prior to use or sale, the constructs may be further processed. For example, they may be processed for storage (e.g. by lyophilization). Alternatively, they may be placed in a suitable solution, usually an aqueous-based solution or carrier with a pH that is suitable for the intended use. For example, for in vivo uses, physiologically acceptable (compatible) carriers/buffers are generally employed.

### Methods

The polypeptides and constructs described herein are excellent reporter sequences and can be used in conjunction with any application known to date for, for example, GFP and other reporter molecules. In addition, they can be advantageously employed in applications where a greater degree or persistence of luminescence is required, being used in many “biological” fields such as the testing of medical samples and medical imaging. However, their uses are not limited to such systems, but can be applied to a wide variety of liquid and/or aqueous-based assays and applications in many fields, including molecular, medical, forensic, military and environmental sciences and engineering. Possible applications include but are not limited to ex vivo visualization at the molecular level in and in vivo imaging. The polypeptides and constructs are especially useful for visualizing/detecting, molecules of interest which are otherwise difficult or impossible to “see”.

Assays and methods of using the polypeptides (without bound metal ions) and constructs (with bound metal ions) include their use to detect and/or visualize one or more molecules of interest. The assays may be continuous or non-continuous. The one or more molecules of interest may be located in an ex vivo sample, or may be in vivo, e.g. within the body of an organism or a subject. The ex vivo methods generally involve a step of contacting the molecule of interest with a polypeptide or construct as described herein. In particular, the polypeptides and constructs that are used for such targeted detection or visualization generally are or comprise fusion or chimeric polypeptides which comprise a targeting moiety that is specific or selective for the molecule of interest. The polypeptides or constructs are generally immobilized on a substrate in a sampling device e.g. in the wells of an assay plate, on beads, on nanoparticles, or microfluidic chip, etc. Alternatively, in some aspects, the molecule of interest is immobilized. The sample is contacted by polypeptides or constructs under conditions that allow (permit) the targeting portion of a polypeptide to come into direct contact with the molecules of interest, the contact being sufficient to allow the targeting portion to bind (chemically attach) to the molecules of interest, thereby forming a complex with the molecule of interest. Binding can be covalent or non-covalent (e.g. ionic, hydrophobic, van der Waals, etc.). Binding can be irreversible or reversible, but if reversible, it is generally sufficiently robust to maintain the attachment, at least through washing steps (if applicable), and while the location of binding is exposed to suitable wavelengths of light and emission from the metals in the constructs is detected.

If the molecules of interest are initially contacted by the polypeptide components of the constructs which comprise a targeting moiety but which do not yet comprise bound metals ions, then one or more washing steps generally ensue to remove unbound polypeptide. Suitable fluorescent metals are then added to the sampling device and they bind to the metal binding sites in the polypeptide.

Once complete complexes of the constructs plus targeted molecules are formed, they are irradiated with (exposed to) one or more wavelengths of light corresponding to (or comprising) the excitation spectrum of the luminescent metals that are chelated within the complexes. If an imaging solution contains ion combinations suitable for upconversion, then an IR source can be used for interrogation. This permits the analysis of opaque biological samples, such as a drop of blood, field samples, or tissue samples, which could be tested for a number of pathogens or protein markers, etc.

Subsequently, luminescence emitted by the chelated fluorescent metals is detected and thus the molecules of interest are detected. If no emission at the indicated wavelengths is detected, then it is concluded that no molecules of interest are present in the sample. In addition, in some aspects, the amount (level, quantity) of luminescence that is detected (measured) is correlated with (indicative of) the amount or level of the molecule of interest that is present in the sample. Those of skill in the art will recognize that such quantitation generally involves the comparison of the detected amount with one or more corresponding predetermined standards or reference values. The reference values are developed prior to testing an unknown sample by using known quantities of the constructs and the molecules of interest. The reference values can include numeric cut-off or threshold values below which no molecule of interest is deemed to be present and above which molecules of interest are deemed to be present. The reference values also generally include a scale or range of values to which a measured value can be compared to quantitate the amount (level, concentration) of molecules of interest in the sample.

In some aspects, the molecules of interest are analytes in an ex vivo sample. Such samples include but are not limited to: serum, plasma, blood, saliva, cerebrospinal fluid, urine, sputum, joint fluid, body cavity fluid, urine, vaginal swabs, feces, whole cells, cell extracts, tissue, biopsy material, aspirates, exudates, slide preparations, fixed cells, solid tumor cells, blood tumor cells, environmental samples, forensic samples, homeland security-related samples and chemical samples. The term “analyte” encompasses any unicellular eukaryotic organism such as yeasts, microalgae and fungi; or prokaryotic organisms such as bacteria; various pathogens (e.g. viruses, bacteria, protozoans, worms, etc.); particular types of cells; cellular components (proteins (e.g. various protein biomarkers), nucleic acids, etc.); small molecules; polymers; etc. In addition, other examples of analytes include but are not limited to: environmental samples, forensic samples, and homeland security-related samples such as explosive ingredients (e.g. gun powder, TNT, plastic bomb components, etc.), narcotic compounds; drugs, especially illegal drugs; organic and biological poisons (e.g. cacodylate, anthrax, influenza virus, etc.); industrial pollutants; and the like. In addition, other examples of analytes include but are not limited to proteins, lipids, membranes, and RNA, DNA samples, vitamins and biological cofactors, etc.

In some aspects, the constructs are used in standard assays involving a fluorescent marker. For example, one or both of a ligand-ligand binding pair can be modified with (e.g. genetically or chemically fused to) a polypeptide of the present invention without disrupting the ability of the two ligands to bind. These and other assays are known in the art and can be adapted for use with the present polypeptides. For example, in some aspects, the antibodies used in ELISA (enzyme linked immunosorbent assay) tests are genetically fused with a protein or polypeptide as described herein (instead of with GFP), and the binding of antigens to the antibodies is measured using a dilute imaging solution of rare earth ions.

In exemplary aspects, the expression and subcellular distribution of the fluorescent proteins within cells can be detected in living tissues without any other experimental manipulation other than to place the cells on a slide and view them through an optical instrument, such as but not limited to, analytical optical instruments, (e.g., a Raman microscope, a confocal microscope, often a fluorescence microscope). This represents a vast improvement over methods of immunodetection that require fixation and subsequent labeling of samples.

### Imaging and Monitoring In Vivo

In other aspects, the constructs are used to detect or image molecules of interest in vivo. This aspect includes non-invasive detection in living organisms, including prokaryotes and eukaryotes. Suitable organisms include but are not limited to bacteria, yeasts, algae, fungi, various protozoans, worms, etc. as well as reptiles and mammals. Exemplary mammals include but are not limited to humans, companion pets and various mammals, especially those of so-called commercial value such as breeding stocks of cattle, horses, chicken, reptiles, amphibians, fishes, worms, etc. Veterinary uses of the technology described herein are encompassed. The in vivo applications may be for research purposes, for diagnostic purposes, for monitoring purposes, or for therapeutic purposes, or for a combination of any of these.

In such aspects, the molecules of interest that are targeted may be at an in vivo site within a body e.g. a tissue, organ or subcellular organelle, including in liquids such as blood, or entities within or on such locations, e.g. disease causing agents such as bacteria and parasites, particular types of cells such as cancer cells, etc. Tissues and organs that may be targeted include but are not limited to: the cardiovascular system: lungs, heart, blood and blood vessels; digestive system components e.g. salivary glands, esophagus, stomach, liver, gallbladder, pancreas, intestines, colon, rectum and anus; components of the endocrine system e.g. endocrine glands such as the hypothalamus, pituitary gland, pineal body or pineal gland, thyroid, parathyroids and adrenals; excretory system components, e.g. kidneys, ureters, bladder and urethra; the lymphatic system e.g. lymph and the nodes and vessels that transport it; the immune system, e.g. tonsils, adenoids, thymus and spleen; muscles; breast tissue; nervous system components e.g. brain, spinal cord, nerves, neural networks; components of the reproductive system e.g. the sex organs, such as ovaries, fallopian tubes, uterus, vulva, vagina, testes, vas deferens, seminal vesicles, prostate and penis; components of the respiratory system e.g. pharynx, larynx, trachea, bronchi, lungs and diaphragm; elements of the skeletal system e.g. bones, cartilage, ligaments and tendons, etc. The target may be an organ or tissue, or a particular aspect thereof, especially an abnormality such as a tumor or abnormal growth, either benign or malignant, etc.

The constructs may be utilized as contrast agents, replacing “dyes” that are currently used. In such aspects, the constructs may aid visualization of organs, tissues, cells, etc. prior to or during surgical procedures, e.g. to visualize damaged heart muscle, monitor reperfusion, visualize nerve damage, for MRI, CAT scans, ultrasound, angiography, echocardiographs, brain scans, etc.

In such aspects, a preparation comprising the constructs in a physiologically compatible carrier (e.g. an aqueous solution or suspension) is administered to a subject in an amount sufficient to result in detectable fluorescence upon irradiation of the location of interest. Thus, the methods described herein may include a step of administering an amount of a polypeptide or construct to a subject, the amount being sufficient to permit detection of one or more molecules of interest within the subject. The polypeptides or constructs are administered in a composition that comprises a physiologically or pharmaceutically acceptable carrier and such compositions are encompassed by the present disclosure. Pharmaceutical compositions generally comprise at least one of the disclosed polypeptides, constructs and/or metals, i.e. one or more than one (a plurality) of different polypeptides, constructs and/or metals (e.g. 2 or more such as 2, 3, 4, 5, 6, 7, 8, 9, 10 or more) may be included in a single formulation. The compositions generally include one or more substantially purified polypeptides, constructs and/or metals as described herein, and a pharmacologically suitable (physiologically compatible) carrier, which may be aqueous or oil-based. In some aspects, such compositions are prepared as liquid solutions or suspensions, or as solid forms such as tablets, pills, powders and the like. Solid forms suitable for solution in, or suspension in, liquids prior to administration are also contemplated (e.g. lyophilized forms of the compounds), as are emulsified preparations. In some aspects, the liquid formulations are aqueous or oil-based suspensions or solutions. In some aspects, the active ingredients are mixed with excipients which are pharmaceutically acceptable and compatible with the active ingredients, e.g. pharmaceutically acceptable salts. Suitable excipients include, for example, water, saline, dextrose, glycerol, ethanol and the like, or combinations thereof. In addition, the composition may contain minor amounts of auxiliary substances such as wetting or emulsifying agents, pH buffering agents, preservatives, and the like. If it is desired to administer an oral form of the composition, various thickeners, flavorings, colorants, diluents, emulsifiers, dispersing aids or binders and the like are added. The compositions of the present invention may contain any such additional ingredients so as to provide the composition in a form suitable for e.g. internal administration. The final amount of polypeptides, constructs and/or metals in a formulations varies, but is generally from about 1-99%. Still other suitable formulations for use in the present invention are found, for example in Remington's Pharmaceutical Sciences, 22nd ed. (2012; eds. Allen, Adejarem Desselle and Felton).

Some examples of materials which can serve as pharmaceutically acceptable carriers include, but are not limited to: ion exchangers, alumina, aluminum stearate, lecithin, serum proteins (such as human serum albumin), buffer substances (such as Tween® 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

“Pharmaceutically acceptable salts” may also be included, e.g. relatively non-toxic, inorganic and organic acid addition salts, and base addition salts, of polypeptides, constructs and/or metals of the present invention. These salts can be prepared in situ during the final isolation and purification of the compounds. In particular, acid addition salts can be prepared by separately reacting purified polypeptides, constructs and/or metals in their free base form with a suitable organic or inorganic acid and isolating the salt thus formed. Those of skill in the art are aware of the large number of such salts, including but not limited to hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, and naphthylate salts. Base addition salts include pharmaceutically acceptable metal and amine salts. Suitable metal salts include the sodium, potassium, calcium, barium, zinc, magnesium, and aluminum salts, among others. See also, for example S. M. Berge, et al., “Pharmaceutical Salts,” J. Pharm. Sci., 66, 1-19 (1977) which is incorporated herein by reference.

The composition may be administered by any suitable route including but not limited to: orally (e.g. as a tablet, troche, pill, capsule, liquid, etc.); intravenously; intraperitoneally; by injection into muscles, organs or tissue to be visualized; by absorption through epithelial or mucocutaneous linings (e.g., nasal, oral, vaginal, rectal, gastrointestinal mucosa, and the like); by inhalation (e.g. as a mist or spray); intravaginally, intranasally, rectally, etc. In preferred embodiments, the mode of administration is intravenous.

The polypeptides, constructs and metals may be delivered via a parenteral carrier system. “Parenteral carrier system” (including variations thereof such as the various specific injectable and infusible dosage forms) refers to compositions comprising one or more pharmaceutically suitable excipients, such as solvents (e.g. water) and co-solvents, solubilizing compounds, wetting compounds, suspending compounds, thickening compounds, emulsifying compounds, chelating compounds, buffers, pH adjusters, antioxidants, reducing compounds, antimicrobial preservatives, bulking compounds, protectants, toxicity adjusters, and special additives.

In some aspects, the technology described herein is used for medical purposes, such as for medical imaging. For example, early diagnosis of tumor malignancy is crucial for timely cancer treatment aimed at imparting desired clinical outcomes. The traditional fluorescence-based imaging for these purposes is unfortunately faced with challenges such as low tissue penetration and background autofluorescence. The constructs described herein overcome these challenges. In particular, upconversion (UC)-based bioimaging as described herein (e.g. using ion combinations) can overcome these limitations because excitation occurs at lower frequencies and the emission at higher frequencies, eliminating overlap and noise. For example, multifunctional silica-based nanocapsules were recently developed which are synthesized to encapsulate two distinct triplet-triplet annihilation UC chromophore pairs. Each nanocapsule emits different colors, blue or green, following a red light excitation. These nanocapsules were further conjugated with either antibodies or peptides to selectively target breast or colon cancer cells, respectively. Both in vitro and in vivo experimental results demonstrated cancer-specific and differential-color imaging from single wavelength excitation as well as far greater accumulation at targeted tumor sites than that due to the enhanced permeability and retention effect. This approach can be applied to host a variety of chromophore pairs i.e. constructs for various tumor-specific, color-coding scenarios and can be employed for diagnosis of a wide range of cancer types within the heterogeneous tumor microenvironment. This approach also advantageously allows for the analysis of opaque biological samples, such as blood, various field samples, etc. without further processing to clarify the samples since, if an imaging solution contains ion combinations suitable for upconversion, then an IR source can be used for interrogation.

Over the past decade the new technical field of super-resolution imaging has emerged. The resolution limit is set by the number of photons a single probe can emit before it photobleaches. In some aspects, the constructs are used as probes that break the current resolution limit since they are not subject to photobleaching.

### Therapeutic Applications

The polypeptides/constructs can also be used to provide therapeutic agents to subjects in need thereof. In general, for such applications, a “cargo” such as a therapeutic agent is attached to the polypeptides described herein and is carried into the body of a subject upon administration. Delivery of the therapeutic agent may be targeted as described elsewhere herein. Advantages are provided since visualization of the construct is possible at the same time the active agent is delivered.

Methods of treating a disease or condition in a subject by administering a therapeutically effective dose of one or more polypeptides and metals or assembled constructs are provided. The term “therapeutically effective dose” (and variations thereof) refers to an amount, dose or dosing regimen of a compound (i.e., active pharmaceutical ingredient, prodrug, or precursor thereof) that is sufficient to treat the disease or condition. Those of skill in the art will recognize that, whereas in some cases, treatment of a disease or condition results in a complete cure (symptoms are eliminated). However, much benefit can also accrue to a subject if symptoms are controlled, lessened or delayed. Suitable doses may vary depending on the form of the compound, the subject's condition, gender, age, ethnicity, and the like, as well as the severity of the symptoms, the route of administration, etc.

Examples of active agents/drugs that can be attached to the polypeptides described herein include but are not limited to: active agents that cause or stimulate apoptosis (killing) of unwanted cells such as cancer cells (e.g. apoptosis promoting agents described in issued U.S. Pat. Nos. 9,657,273, 8,831,738, 8,247,380, 7,786,275 and references disclosed therein); biologically active agents such as, for example, hypnotics and sedatives, psychic energizers, tranquilizers, respiratory drugs, anticonvulsants, muscle relaxants, antiparkinson agents (dopamine antagnonists), analgesics, anti-inflammatories, antianxiety drugs (anxiolytics), appetite suppressants, antimigraine agents, muscle contractants, anti-infectives (antibiotics, antivirals, antifungals, vaccines) antiarthritics, antimalarials, antiemetics, anepileptics, bronchodilators, cytokines, growth factors, anti-cancer agents, antithrombotic agents, antihypertensives, cardiovascular drugs, antiarrhythmics, antioxicants, anti-asthma agents, hormonal agents including contraceptives, sympathomimetics, diuretics, lipid regulating agents, antiandrogenic agents, antiparasitics, anticoagulants, neoplastics, antineoplastics, hypoglycemics, nutritional agents and supplements, growth supplements, antienteritis agents, vaccines, antibodies, diagnostic agents, and contrasting agents.

The active agent may fall into one of a number of structural classes, including but not limited to small molecules (preferably insoluble small molecules), peptides, polypeptides, proteins, antibodies, antibody fragments, polysaccharides, steroids, nucleotides, oligonucleotides, polynucleotides, fats, electrolytes, and the like. Preferably, an active agent for coupling to a polymer as described herein possesses a native amino group, or alternatively, is modified to contain at least one reactive amino group suitable for conjugating to a polymer described herein.

Specific examples of active agents suitable for covalent attachment include but are not limited to agalsidase, alefacept, aspariginase, amdoxovir (DAPD), antide, becaplermin, calcitonins, cyanovirin, denileukin diftitox, erythropoietin (EPO), EPO agonists (e.g., peptides from about 10-40 amino acids in length and comprising a particular core sequence as described in WO 96/40749), dornase alpha, erythropoiesis stimulating protein (NESP), coagulation factors such as Factor V, Factor VII, Factor Vila, Factor VIII, Factor IX, Factor X, Factor XII, Factor XIII, von Willebrand factor; ceredase, cerezyme, alpha-glucosidase, collagen, cyclosporin, alpha defensins, beta defensins, desmopressin, exedin-4, granulocyte colony stimulating factor (GCSF), thrombopoietin (TPO), alpha-1 proteinase inhibitor, elcatonin, granulocyte macrophage colony stimulating factor (GMCSF), fibrinogen, filgrastim, growth hormones human growth hormone (hGH), somatropin, growth hormone releasing hormone (GHRH), GRO-beta, GRO-beta antibody, bone morphogenic proteins such as bone morphogenic protein-2, bone morphogenic protein-6, OP-1; acidic fibroblast growth factor, basic fibroblast growth factor, CD-40 ligand, heparin, human serum albumin, low molecular weight heparin (LMWH), interferons such as interferon alpha, interferon beta, interferon gamma, interferon omega, interferon tau, consensus interferon; interleukins and interleukin receptors such as interleukin-1 receptor, interleukin-2, interleukin-2 fusion proteins, interleukin-1 receptor antagonist, interleukin-3, interleukin-4, interleukin-4 receptor, interleukin-6, interleukin-8, interleukin-12, interleukin-13 receptor, interleukin-17 receptor; lactoferrin and lactoferrin fragments, luteinizing hormone releasing hormone (LHRH), insulin, pro-insulin, insulin analogues (e.g., mono-acylated insulin as described in U.S. Pat. No. 5,922,675), amylin, C-peptide, somatostatin, somatostatin analogs including octreotide, vasopressin, follicle stimulating hormone (FSH), influenza vaccine, insulin-like growth factor (IGF), insulintropin, macrophage colony stimulating factor (M-CSF), plasminogen activators such as alteplase, urokinase, reteplase, streptokinase, pamiteplase, lanoteplase, and teneteplase; nerve growth factor (NGF), osteoprotegerin, platelet-derived growth factor, tissue growth factors, transforming growth factor-1, vascular endothelial growth factor, leukemia inhibiting factor, keratinocyte growth factor (KGF), glial growth factor (GGF), T Cell receptors, CD molecules/antigens, tumor necrosis factor (TNF), monocyte chemoattractant protein-1, endothelial growth factors, parathyroid hormone (PTH), glucagon-like peptide, somatotropin, thymosin alpha 1, rasburicase, thymosin alpha 1 IIb/IIIa inhibitor, thymosin beta 10, thymosin beta 9, thymosin beta 4, alpha-1 antitrypsin, phosphodiesterase (PDE) compounds, VLA-4 (very late antigen-4), VLA-4 inhibitors, bisphosponates, respiratory syncytial virus antibody, cystic fibrosis transmembrane regulator (CFTR) gene, deoxyreibonuclease (Dnase), bactericidal/permeability increasing protein (BPI), and anti-CMV antibody. Exemplary monoclonal antibodies include etanercept (a dimeric fusion protein consisting of the extracellular ligand-binding portion of the human 75 kD TNF receptor linked to the Fc portion of IgG), abciximab, adalimumab, afelimomab, alemtuzumab, antibody to B-lymphocyte, atlizumab, basiliximab, bevacizumab, biciromab, bertilimumab, CDP-571, CDP-860, CDP-870, cetuximab, clenoliximab, daclizumab, eculizumab, edrecolomab, efalizumab, epratuzumab, fontolizumab, gavilimomab, gemtuzumab ozogamicin, ibritumomab tiuxetan, infliximab, inolimomab, keliximab, labetuzumab, lerdelimumab, olizumab, radiolabeled lym-1, metelimumab, mepolizumab, miturnomab, muromonad-CD3, nebacumab, natalizumab, odulimomab, omalizumab, oregovomab, palivizumab, pemtumomab, pexelizumab, rhuMAb-VEGF, rituximab, satumomab pendetide, sevirumab, siplizumab, tositumomab, .sup.131tositumomab, trastuzumab, tuvirumab and visilizumab.

Additional agents suitable for attachment include, but are not limited to: tacrine, memantine, rivastigmine, galantamine, donepezil, levetiracetam, repaglinide, atorvastatin, alefacept, tadalafil, vardenafil, sildenafil, fosamprenavir, oseltamivir, valacyclovir and valganciclovir, abarelix, adefovir, alfuzosin, alosetron, amifostine, amiodarone, aminocaproic acid, aminohippurate sodium, aminoglutethimide, aminolevulinic acid, aminosalicylic acid, amlodipine, amsacrine, anagrelide, anastrozole, aprepitant, aripiprazole, asparaginase, atazanavir, atomoxetine, anthracyclines, bexarotene, bicalutamide, bleomycin, bortezornib, buserelin, busulfan, cabergoline, capecitabine, carboplatin, carmustine, chlorambucin, cilastatin sodium, cisplatin, cladribine, clodronate, cyclophosphamide, cyproterone, cytarabine, camptothecins, 13-cis retinoic acid, all trans retinoic acid; dacarbazine, dactinomycin, daptomycin, daunorubicin, deferoxamine, dexarnethasone, diclofenac, diethylstilbestrol, docetaxel, doxorubicin, dutasteride, eletriptan, emtricitabine, enfuvirtide, eplerenone, epirubicin, estramustine, ethinyl estradiol, etoposide, exemestane, ezetimibe, fentanyl, fexofenadine, fludarabine, fludrocortisone, fluorouracil, fluoxymesterone, flutamide, fluticazone, fondaparinux, fulvestrant, gamma-hydroxybutyrate, gefitinib, gemcitabine, epinephrine, L-Dopa, hydroxyurea, icodextrin, idarubicin, ifosfamide, imatinib, irrinotecan, itraconazole, goserelin, laronidase, lansoprazole, letrozole, leucovorin, levamisole, lisinopril, lovothyroxine sodium, lomustine, mechlorethamine, medroxyprogesterone, megestrol, melphalan, memantine, mercaptopurine, mequinol, metaraminol bitartrate, methotrexate, metoclopramide, mexiletine, miglustat, mitomycin, mitotane, mitoxantrone, modafinil, naloxone, naproxen, nevirapine, nicotine, nilutamide, nitazoxanide, nitisinone, norethindrone, octreotide, oxaliplatin, palonosetron, pamidronate, pemetrexed, pergolide, pentostatin, pilcamycin, porfimer, prednisone, procarbazine, prochlorperazine, ondansetron, palonosetron, oxaliplatin, raltitrexed, rosuvastatin, sirolimus, streptozocin, pimecrolimus, sertaconazole, tacrolimus, tamoxifen, tegaserod, temozolomide, teniposide, testosterone, tetrahydrocannabinol, thalidomide, thioguanine, thiotepa, tiotropium, topiramate, topotecan, treprostinil, tretinoin, valdecoxib, celecoxib, rofecoxib, valrubicin, vinblastine, vincristine, vindesine, vinorelbine, voriconazole, dolasetron, granisetron, formoterol, fluticasone, leuprolide, midazolam, alprazolam, amphotericin B, podophylotoxins, nucleoside antivirals, aroyl hydrazones, sumatriptan, eletriptan; macrolides such as erythromycin, oleandomycin, troleandomycin, roxithromycin, clarithromycin, davercin, azithromycin, flurithromycin, dirithromycin, josamycin, spiromycin, midecamycin, loratadine, desloratadine, leucomycin, miocamycin, rokitamycin, andazithromycin, and swinolide A; fluoroquinolones such as ciprofloxacin, ofloxacin, levofloxacin, trovafloxacin, alatrofloxacin, moxifloxicin, norfloxacin, enoxacin, gatifloxacin, gemifloxacin, grepafloxacin, lomefloxacin, sparfloxacin, temafloxacin, pefloxacin, amifloxacin, fleroxacin, tosufloxacin, prulifloxacin, irloxacin, pazufloxacin, clinafloxacin, and sitafloxacin; aminoglycosides such as gentamicin, netilmicin, paramecin, tobramycin, amikacin, kanamycin, neomycin, and streptomycin, vancomycin, teicoplanin, rampolanin, mideplanin, colistin, daptomycin, gramicidin, colistimethate; polymixins such as polymixin B, capreomycin, bacitracin, penems; penicillins including penicllinase-sensitive agents like penicillin G, penicillin V; penicillinase-resistant agents like methicillin, oxacillin, cloxacillin, dicloxacillin, floxacillin, nafcillin; gram negative microorganism active agents like ampicillin, amoxicillin, and hetacillin, cillin, and galampicillin; antipseudomonal penicillins like carbenicillin, ticarcillin, azlocillin, mezlocillin, and piperacillin; cephalosporins like cefpodoxime, cefprozil, ceftbuten, ceftizoxime, ceftriaxone, cephalothin, cephapirin, cephalexin, cephradrine, cefoxitin, cefamandole, cefazolin, cephaloridine, cefaclor, cefadroxil, cephaloglycin, cefuroxime, ceforanide, cefotaxime, cefatrizine, cephacetrile, cefepime, cefixime, cefonicid, cefoperazone, cefotetan, cefimetazole, ceftazidime, loracarbef, and moxalactam, monobactams like aztreonam; and carbapenems such as imipenem, meropenem, and ertapenem, pentamidine isetionate, albuterol sulfate, lidocaine, metaproterenol sulfate, beclomethasone diprepionate, triamcinolone acetamide, budesonide acetonide, salmeterol, ipratropium bromide, flunisolide, cromolyn sodium, and ergotamine tartrate; taxanes such as paclitaxel; SN-38, tyrphostines, aminohippurate sodium, amphotericin B, doxorubicin, aminocaproic acid, aminolevulinic acid, aminosalicylic acid, metaraminol bitartrate, pamidronate di sodium, daunorubicin, levothyroxine sodium, lisinopril, cilastatin sodium, mexiletine, cephalexin, deferoxamine, and amifostine.

Exemplary peptides or proteins for coupling to a polypeptide as described herein include Erythropoietin (EPO), IFN-α, IFN-β, consensus IFN, Factor VIII, B-domain deleted factor VIII, Factor IX, Granulocyte-colony stimulating factor (GCSF), Granulocyte-macrophage colony-stimulating factor (GM-CSF), hGH, insulin, Follicle-stimulating hormone (FSH), peptides having GLP-1 activity, desmopressin, amdoxivir, and Parathyroid hormone (PTH).

### Detection

Any suitable method of detecting the luminescence emitted by the luminescent metals described herein may be used in the present practices. Examples of detection methods and systems include but are not limited to: flow cytometers, fluorescent correlation spectrometry, single particle microscopy, detection using filter fluorometers, spectrofluorometers, spectroscopic microscopes, phosphoroscopes, and fluorescence microscopes. In particular, for ex vivo imaging and detection the following detection methods are generally used spectroflurometers, filter fluoremeters, flow cytometry, and fluorescence imaging; whereas for in vivo imaging, the following detection methods are generally used fluorescence microscopy, microfluidics and flow cytometry. Any method of detection that detects the requisite wavelengths of light energy can be used.

### Kits

Test kits for ex vivo use typically comprise containers to contain the constructs as described herein, either in solution or in a dried (e.g. lyophilized) form; or containers to separately contain disclosed polypeptides and metals that bind thereto, both or either of which may be supplied in a solution or in a dried (e.g. lyophilized) form. The constructs or polypeptides may be attached to a substrate, e.g. beads such as magnetic beads, etc. and various solutions for their use may be included, as well as control samples, instructions, etc. Examples of test kit formats are described, for example, in issued U.S. Pat. Nos. 9,897,601 and 9,891,205, the complete contents of which are hereby incorporated by reference, and in references cited therein. Control reagents and instructions for use may also be included. Considering the very low detection limits exhibited by the present constructs, test kits can advantageously be miniaturized and sample volume and/or concentration requirements can be decreased, compared to prior art test kits. For example, because the proteins/polypeptide chelated with luminescent metals described herein do not bleach (fade over time) much less is needed to detect an analyte and therefore quantities in the milliliter range can be decreased to the microliter range

For medical applications, kits comprising therapeutic components may comprise containers of i) constructs or ii) polypeptides and metals in solution or in a dry form suitable for reconstitution prior to use. In this case, the solutions are physiologically suitable for internal administration to a subject.

### Other Applications

Other applications of the present technology include but are not limited to: use as markers for detecting specific nucleic acids and/or proteins; for detecting gene expression; for measuring the mass transport of proteins, as super resolution probes for homeland security or forensic sciences, etc. In addition, the polypeptide or construct can be attached to anti-sense DNA/RNA to detect the existence of specific DNA or RNA.

With respect to the use in detecting gene expression, the polypeptides and/or constructs are used as follows: The gene of interest is expressed as a fusion protein with a polypeptide as described herein, and the expression pattern in cells or tissues is then monitored, e.g. by con-focal microscope taking advantage of luminescence of the construct. Alternatively, polypeptide-complexed antibody against the target polypeptides can be applied to the fixed cell or tissues to detect the expression pattern of the target molecules.

Unless otherwise indicated, numbers expressing quantities of ingredients, constituents, reaction conditions and so forth used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements. Where a range of values is provided, it is understood that each intervening value between the upper and lower limit of that range (to a tenth of the unit of the lower limit) is included in the range and encompassed within the invention, unless the context or description clearly dictates otherwise. In addition, smaller ranges between any two values in the range are encompassed, unless the context or description clearly indicates otherwise.

It should be emphasized that the above-described embodiments and following specific examples of the present invention, particularly, any “preferred” embodiments, are merely possible examples of implementations, merely set forth for a clear understanding of the principles of the invention. Many variations and modifications may be made to the above described embodiment(s) of the invention without departing substantially from the spirit and principles of the invention. All such modifications and variations are intended to be included herein within the scope of this disclosure and the present invention and protected by the following claims. It is to be understood that this invention is not limited to particular embodiments described herein above and below, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this invention belongs. Representative illustrative methods and materials are herein described; methods and materials similar or equivalent to those described herein can also be used in the practice or testing of the present invention.

All publications and patents cited in this specification are herein incorporated by reference as if each individual publication or patent were specifically and individually indicated to be incorporated by reference, and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual dates of public availability and may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent”, or “except for [a particular feature or element]”, or “wherein [a particular feature or element] is not present (included, etc.) . . . ”.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### EXAMPLES

### Example 1

State-of-the-art fluorescent expression probes, such as GFP and others, have revolutionized biotechnology but continue to have serious limitations in terms of performance and spectroscopic range (both in time and wavelength). In particular, these probes photobleach after a few seconds of exposure, have limited excitation and luminescence range, and misfolding of the proteins upon cellular expression renders the protein completely nonfluorescent.

Calsequestrin is a highly acidic and conserved Ca²⁺ storage/buffer protein existing in both skeletal and cardiac muscles of every mammal and contains up to 60 Ca²⁺ binding sites. Rare earth cations are smaller in size than Ca²⁺ and can carry a higher charge. The result is that calsequestrin has a much higher affinity for rare earths than it does for Ca²⁺. The fact that calsequestrin has 60 binding sites means that each protein can accommodate ˜60 independent luminescent metal emitters and thus rival the luminescence intensity of common organic fluorophores and fluorescent proteins such as Green Fluorescent Protein (GFP).

Several calsequestrin variants were developed and tested. Experiments with various metals showed that metal ions bind quantitatively and irreversibly to the variants, and that even at the 1 nM level fluorescence was well above background, including when variants were expressed in live cells. Some metal ions were detected in the pM range and possibly at the single molecule level, as described in detail below.

This rare earth-binding platform advantageously does not suffer from prior art limitations. Its luminescence properties are completely independent of small protein misfolds (the variants fold and unfold properly in the presence or absence of cations), the excited rare earth elements do not photobleach or photoblink and the luminescence wavelength is dependent only on the rare earth elements used and can thus be “tuned” to desired wavelengths by varying the composition.

### Example 1. Characterization of Calsequestrin Variants

### Methods

### Recombinant Protein Expression and Purification.

The cDNA corresponding to the variant genes was cloned into pET30a vector for overexpression. For expression of recombinant protein, 200 mL of Luria-Bertani medium containing 100 μg mL⁻¹ kanamycin and 34 μg mL⁻¹ chloramphenicol were inoculated with a freezer stock of Rosetta cells (EMD Millipore, Billerica, Mass.) containing the pET30a-variant construct and grown overnight at 37° C. while shaking. This culture was used to inoculate 3 L of Luria-Bertani medium, which was grown to an OD₆₀₀ of 0.4 at 37° C. with shaking. The cells were then brought to 18° C. with continuous shaking, and isopropyl β-thio-galactopyranoside was added to a final concentration of 0.2 mM. The culture was grown at 18° C. while shaking for an additional 24 hrs. Cells were collected by centrifugation at 5,000 rpm for 20 min at 4° C. The cell pellet was resuspended in 40 mL lysis buffer (50 mM Sodium phosphate, pH 8.0, 300 mM NaCl and, 15 mM imidazole) and was sonicated five times with 15-s pulses (model 450 sonifier; Branson Ultrasonics, Danbury, Conn.). The lysate was cleared by centrifugation at 16,000 rpm for 25 min. Cleared supernatant was applied to 15 mL nickel-nitrilotriacetate agarose (Qiagen, Germantown, Md.), equilibrated with lysis buffer, and placed into a gravity-flow column. The column was washed with 20 column-volumes washing buffer (50 mM Sodium phosphate, pH 8.0, 300 mM NaCl, and 25 mM imidazole), and protein was eluted with elution buffer (50 mM Sodium phosphate, pH 8.0, 300 mM NaCl, and 250 mM imidazole). Column fractions containing variant protein were desalted and concentrated into buffer A (20 mM Tris, pH 8.0, 5% v/v glycerol) using an Amicon 8050 ultrafiltration cell with a 10-kDa cutoff membrane (Millipore). Concentrated protein was applied to a Mono-Q column (GE Healthcare) that was pre-equilibrated with buffer A using a flow rate of 2 mL min-. Variant polypeptides were eluted from the column with a linear NaCl gradient. The fractions containing variant polypeptides were pooled and buffer exchanged into 20 mM Tris, pH 8.0. All expression and purification of variants was performed in an identical manner with SDS-PAGE to check the presence and purity of enzymes after each purification step. Protein concentrations were determined by using BCA assay kit (Thermo Fisher Scientific).

Site-directed mutations were created in the calsequentrin coding region by PCR-based amplification using Phusion High-Fidelity DNA polymerase (New England Biolabs, Ipswich, Mass.). The amplification was performed using complementary plus- and minus-strand oligonucleotides containing the target mutations, and was followed by DpnI (New England Biolabs, Ipswich, Mass.) digestion to degrade the template prior to transformation of competent E. coli Rosetta cells (EMD Millipore, Billerica, Mass.). Both mutations were confirmed by DNA sequencing (GENEWIZ, Plainfield, N.J.).

### Spectroscopy.

All steady state emission spectra were collected with a home-built luminescence spectrometer that utilized either a doubled Ti:Sapphire laser (450 nm-350 nm) or a Hg:Xe arc lamp that was passed through a ¼ meter spectrometer before exciting the sample. The sample holder was fixed at the focal point of the collection optics and light emitted by the samples was collected at a 90° angle from the excitation beam. Scatter from the excitation beam into the monochromator was removed with a 33 M KNO₂ water filter (OD>6 at λₑₓ<408 nm). The emitted light was then dispersed by an Acton 500i monochromator and detected with a thermoelectrically cooled Hamamatsu R943-02 photomultiplier tube. The detector signal was then passed through a wide-band preamplifier (SRS model SR445) and fed to a photon counter (SRS model SR400). Data was transferred to a PC for further manipulation using Igor Pro software (version 6.34, WaveMetrics Inc.). All spectra were an average of a least three scans, corrected against an Ocean Optics calibrated light source (model HL3-INT-CAL, spectral irradiance standard) and background subtracted. Phosphorescence lifetimes were measured with the same apparatus, but light was collected by a 1 sec gate that was scanned after the excitation light was turned off. For excitation experiments, the emission spectrometer was set to the maximum phosphorescence intensity and the excitation spectrometer was scanned from 315 nm-550 nm or from 315 nm-500 nm. A 500 nm or 550 nm cutoff filter in conjunction with the KNO₂ water filter was placed between the sample and emission spectrometer in order to eliminate scatter for the excitation beam.

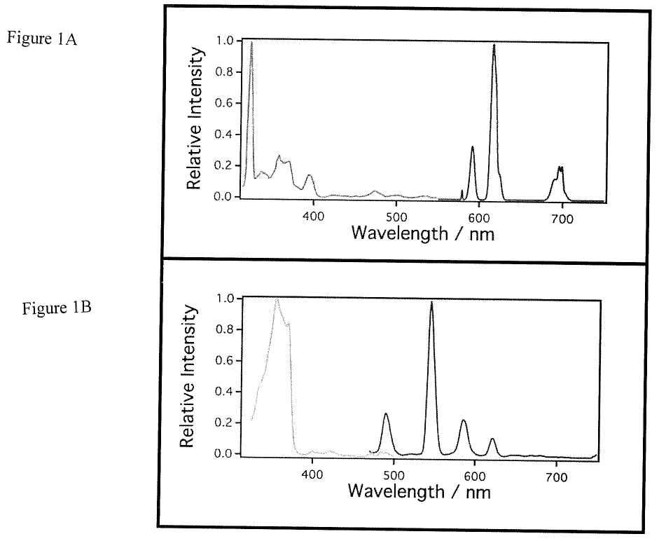

Depicted in FIG. 1A and FIG. 1B are the excitation and phosphorescence spectra of Variant-615 and Variant-544, respectively. The excitation spectra display a number of excitation bands of varying corresponding to a number of spin forbidden excitations associated with the metal ions themselves. In solution, Eu³⁺ and Tb³⁺ are near completely non-luminescent due to the symmetry around the solvated ions and the strong vibrational coupling of between the metal ions and water molecules. When the metals are bound to variants they become highly phosphorescent with transitions corresponding to the well-known spin forbidden states of each ion.

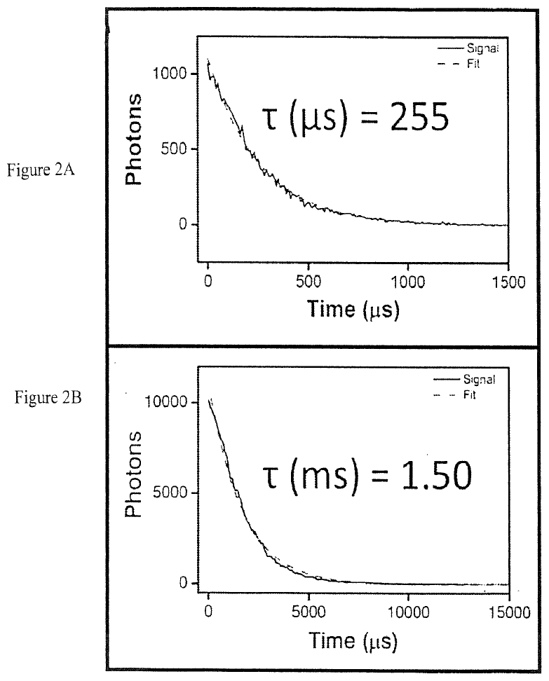

FIGS. 2A and 2B depict the phosphorescence decay curves of Variant-615 and Variant-544, respectively. Unlike fluorescent proteins and small fluorescent organic molecules which possess fluorescence decays in the nanosecond time regime, Variant-615 and Variant-544, respectively, have luminescence decays in the 100 microseconds to millisecond time scale. This is a confirmation of the phosphorescent nature of the luminescence signal.

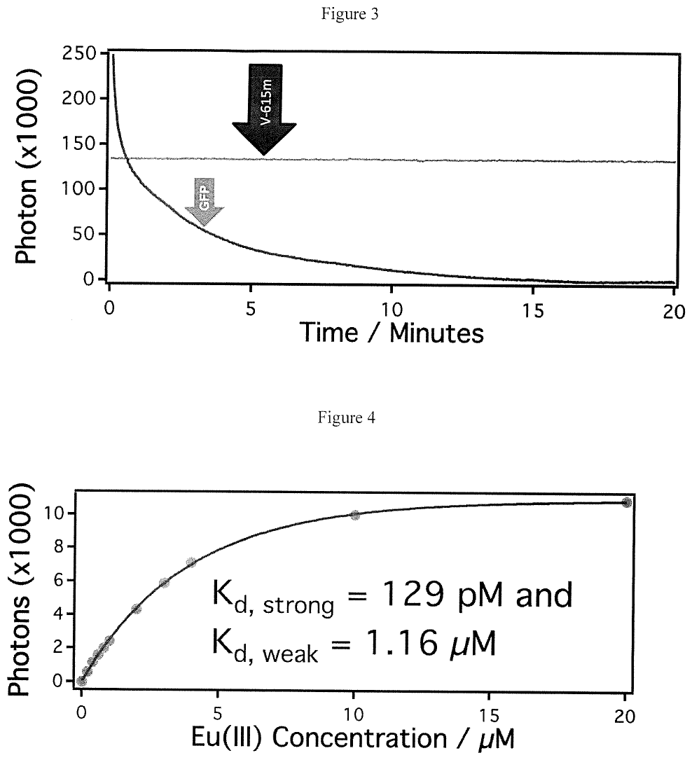

FIG. 3 is a head-to-head comparison of the photostability of GFP vs. Variant-615. In this experiment the laser power was adjusted such that the amount of light absorbed at 395 nm was the same for each sample. At the start of the experiment GFPs intensity was significantly higher than that of Variant-615 but GFP photodegraded quickly. In contrast, Variant-615 displayed virtually no photodegradation even after an hour of continuous laser illumination. After 1-2 seconds of laser irradiation, the luminescence intensity of Variant 615 was greater than that observed in GFP.

FIG. 4 depicts the results of a titration study in which a 112 nM solution of a variant was treated with increasing concentrations of Eu²⁺. Since Eu²⁺ does not emit light in solution, the amount of bound Eu²⁺ was determined by plotting the intensity of 615 nm peak vs. Eu²⁺ concentration. The data was fit to a binding model in which the variant contained 4 strong binding sites and 36 weaker ones. This analysis demonstrates that this variant has four very strong binding sites (K_{d, strong}=129 μM) and multiple weaker ones (K_{d, strong}=1.16 μM).

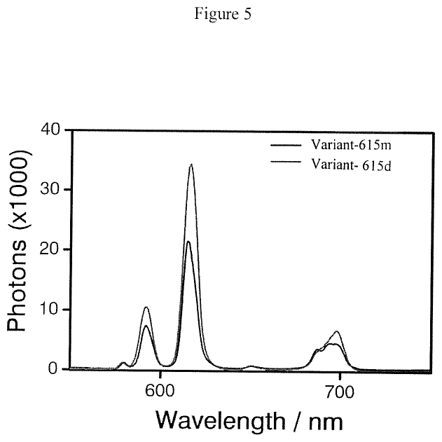

The variants can be engineered to have any number of rare-earth binding sites. As an example, Variant-615d has been engineered to have twice the number of binding as that of Variant-615m. FIG. 5 clearly shows that the luminescence intensity scales nearly linearly with the number of binding sites on the engineered protein.

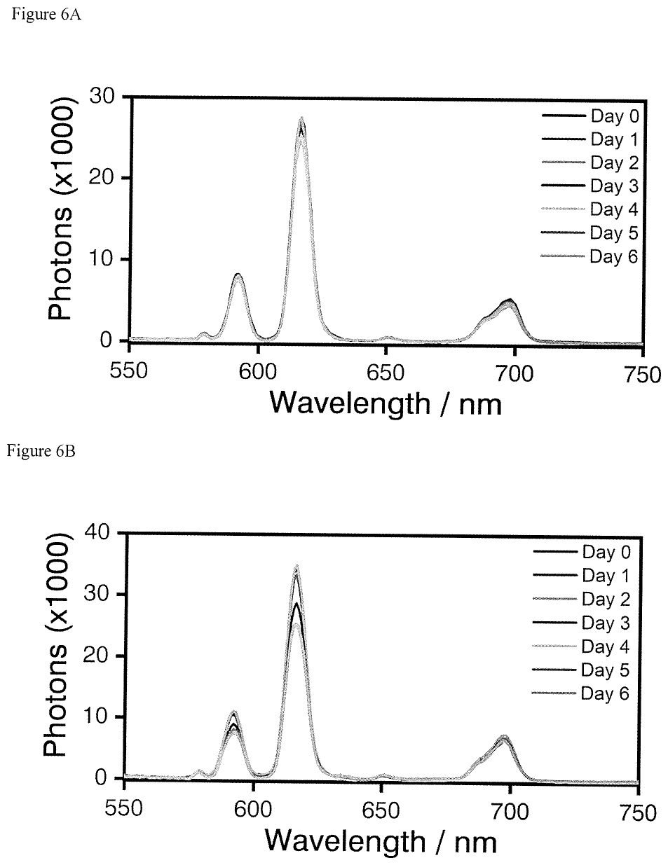

The variants can be stored long-term as a lyophilized powder or as a glycerol stock solution at −80 K. A study was conducted to probe the short-term shelf-life of samples after they have been prepared for used. In this study samples of Variant-615m were prepared in HEPES buffer at pH 7.4 and allows to sit on the bench top for 6 days and in the refrigerator for 6 days. The sample stored in the refrigerator shown no discernable degradation. Samples left on the beach top degraded by 26%. The results are depicted in FIGS. 6A and 6B.

Fluorescence microscopy. All fluorescence imaging measurements were made using an Olympus IX71 inverted fluorescence microscope fitted with a Hg:Xe lamp. Light from the lamp was passed through a 420 nm bandpass filter (40 nm FWHM; Chroma Technologies; AT420/40X), reflected through a microscope objective (Olympus Apo 100×1.45 N/A) with a dichroic mirror (Chroma Technologies; AT455DC) and focused onto the sample. The emitted light was collected by the objective, passed through the dichroic mirror, and then passed through either a 545 nm longpass filter before being imaged onto a Hamamatsu ORCAII CCD camera. All data was collected using the Advanced Metamorph software suite (Olympus, Inc). FIG. 7 depicts a phosphorescence micrograph of E. coli. that are expressing Variant-615m. In this study, the transfected E. coli. was incubated with 0.1 mM Eu³⁺ in the culture media. The presence of the metal ion had no effect on the growth of the E. coli. Transfected samples that where mineralized into Variant 615m displace bright phosphorescence in living E. coli. and could be easily imaged (FIG. 7). The luminosity of Variant-615m provided a high contrast image. Samples prepared with E. coli. that were cultured with 0.1 mM Eu³⁺ but not transfected with Variant-615m did not display enough phosphorescence to be detected above the weak autofluorescence of sample.

### Example 3. Fluorescence Upconversion

### In Lyophilized Preparations

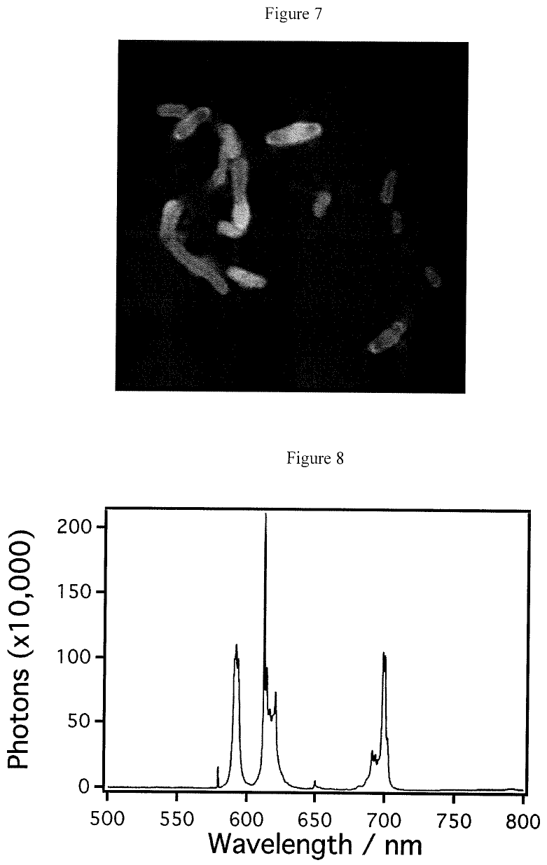

Further experiments determined the luminescence spectrum of lyophilized Variant 980m chelated with Y³⁺, Yb³⁺ and Eu⁺³ when excited with two photons of 980 nm near IR light produced with a continuous wave (cw)-diode laser. Eu³⁺ accounted for 2% of the sample and is the emitting center. The results are presented in FIG. 8. As can be seen, luminescence upconversion results from excitation with 980 nm light showing that chelation by the subject variant polypeptides does not have a deleterious effect on the exciton hopping process.

In short, these samples show a strong upconversion signal

### In Liquid Preparations

Conditions for upconversion in liquid samples are developed. These include developing the optimal conditions for photon capture by a photosensitizing metal ion and metal centers that serve as centers for phosphorescence in the visible region of the electromagnetic spectrum.

In addition, engineered polypeptides/proteins are developed in which a greater number of photosensitizing metal ions complex the weaker chelation sites, allowing for exciton hopping to be “funneled” to the phosphorescent centers located in the strong binding sites.

### Example 4. Variants with Functional Groups

Variants have been prepared with attached functional groups (e.g. biotin, maleimide).

Experiments conducted with the Variant-615m-biotin construct showed no loss in phosphorescence intensity and the construct bound tightly to streptavidin in analytical testing.

Tests of the reactivity of Variant 615m-maleimide toward cysteine residues in proteins showed that the constructs are stable and form covalent bonds with cysteine residues in protein.

While the invention has been described in terms of its several exemplary embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A construct comprising,
i) a genetically engineered, recombinant polypeptide comprising a plurality of luminescent metal binding sites, and
ii) a plurality of chelated luminescent metals.

2. The construct of claim 1, wherein the chelated luminescent metals are rare earth metals or actinides.

3. The construct of claim 2, wherein the chelated luminescent metals are selected from the group consisting of Ce, Dy, Er, Eu, Gd, Ho, La, Lu, Nd, Pr, Pm, Sm, Sc, Tb, Tm, Yb, and Y.

4. The construct of claim 2, wherein the actinides are U or Th.

5. The construct of claim 1, wherein the construct comprises at least two different types of chelated luminescent metals.

6. The construct of claim 1, wherein the plurality of luminescent metal binding sites comprise
i) one or more high affinity luminescent metal-binding sites; and
ii) one or more medium affinity luminescent metal-binding sites.

7. The construct of claim 1, wherein the genetically engineered, recombinant polypeptide is a modified calcium binding polypeptide.

8. The construct of claim 1, wherein the genetically engineered, recombinant polypeptide is a fusion polypeptide.

9. The construct of claim 8, wherein the fusion polypeptide comprises a targeting moiety and/or a half-life expanding moiety.

10. The construct of claim 9, wherein the targeting moiety is an antibody or antigen binding portion thereof.

11. A genetically engineered, recombinant polypeptide comprising a plurality of luminescent metal binding sites.

12. The genetically engineered, recombinant polypeptide of claim 11, wherein the plurality of luminescent metal binding sites comprise
i) one or more high affinity luminescent metal-binding sites; and
ii) one or more medium affinity luminescent metal-binding sites.

13. The genetically engineered, recombinant polypeptide of claim 11, wherein the genetically engineered, recombinant polypeptide is a modified calcium binding polypeptide.

14. The genetically engineered, recombinant polypeptide of claim 11, wherein the genetically engineered, recombinant polypeptide is a fusion polypeptide.

15. The genetically engineered, recombinant polypeptide of claim 14, wherein the fusion polypeptide comprises a targeting moiety and/or a half-life expanding moiety.

16. The genetically engineered, recombinant fusion polypeptide of claim 15, wherein the targeting moiety is an antibody or antigen binding portion thereof.

17. A nucleic acid encoding the genetically engineered, recombinant polypeptide of claim 11.

18. A plasmid comprising the nucleic acid of claim 17.

19. A cell comprising the plasmid of claim 18.

20. A detection method, comprising:
combining a sample comprising an analyte with one or more polypeptides or proteins each having chelated thereto one or more luminescent metals;
binding a molecule of interest in said sample with said one or more polypeptides or proteins;
exciting said one or more luminescent metals with electromagnetic energy; and
detecting luminescence from said one or more luminescent metals after said step of exciting.

21. The detection method of claim 20, wherein one or more of the steps occurs on a chip or in a microwell device.

22. The detection method of claim 20, wherein one or more of the steps are performed as part of an ELISA assay.

23. The method of claim 20, wherein the sample is selected from the group consisting of serum, plasma, blood, saliva, cerebrospinal fluid, urine, sputum, joint fluid, body cavity fluid, whole cells, cell extracts, tissue, biopsy material, aspirates, exudates, slide preparations, fixed cells, solid tumor cells, blood tumor cells, environmental samples, forensic samples, homeland security-related samples and chemical samples.

24. The method of claim 20, wherein the analyte is a protein, an amino acid, a peptide, a nucleic acid, carbohydrate, lipid, vitamin, hemoglobin, explosive chemicals or remnants thereof, poisons, virus, bacteria or any target molecules in medical diagnostic assay, an anti-terrorism assay target, or a forensic assay target.

25. The method of claim 20, wherein the step of detecting is performed by Förster Resonance Energy Transfer (FRET), enzyme linked immunosorbent assay (ELISA) testing, flow cytometry, fluorescent correlation spectrometry or single particle microscopy.

26. A method of detecting an analyte located in the body of a subject, comprising
administering to the subject a composition comprising the construct of claim 1,
irradiating the subject with electromagnetic energy to excite the one or more luminescent metals in the construct; and
detecting luminescence from said one or more luminescent metals after said step of irradiating.

27. The method of claim 26, wherein the subject is a cancer patient and the analyte is a tumor cell marker.
